(19) United States
(12) Patent Application Publication
(10) Pub. No.: US 2024/0092883 A1
(43) Pub. Date: Mar. 21, 2024

(54) METHODS OF PURIFYING RANIBIZUMAB OR A RANIBIZUMAB VARIANT

(71) Applicant: Coherus Biosciences, Inc., Redwood City, CA (US)

(72) Inventors: John Robert Ogez, Longmont, CO (US); Brian L. Williamson, Camarillo, CA (US); Nessa Mary Noone Hawkins, Redwood City, CA (US)

(73) Assignee: Coherus Biosciences, Inc., Redwood City, CA (US)

(21) Appl. No.: 17/768,106

(22) PCT Filed: Oct. 9, 2020
(86) PCT No.: PCT/US2020/055008

§ 371 Date: Apr. 11, 2022

Related U.S. Application Data
(60) Provisional application No. 62/914,307, filed on Oct. 11, 2019.

(51) Int. Cl. C07K 16/22

(52) U.S. Cl. CPC C07K 16/22; C07K 2317/24; C07K 2317/76

(57) ABSTRACT

Provided herein are processes for manufacturing recombinant ranibizumab or a ranibizumab variant that include providing a liquid comprising recombinant ranibizumab or a ranibizumab variant that is substantially free of cells; capturing the recombinant ranibizumab or the ranibizumab variant in the liquid using an affinity chromatography column, wherein the eluate of the affinity chromatography column comprises the recombinant ranibizumab or the ranibizumab variant; purifying the recombinant ranibizumab or the ranibizumab variant in the eluate of step (b) using a first cation exchange chromatography column and buffers that have a pH of about pH 5.5 to about 7.5, wherein the eluate from the first cation exchange chromatography column comprises the recombinant ranibizumab or the ranibizumab variant; and purifying the recombinant ranibizumab or the ranibizumab variant in the eluate from the first cation exchange chromatography column using a second cation chromatography column and buffers that have a pH of about pH 4.0 to about pH 5.4, wherein the eluate from the second cation chromatography column comprises the recombinant ranibizumab or the ranibizumab variant.

### CROSS-REFERENCE TO RELATED APPLICATION

This application claims priority to U.S. Provisional Patent Application Ser. No. 62/914,307, filed Oct. 11, 2019, the entire contents of which are herein incorporated by reference.

### TECHNICAL FIELD

This invention relates to methods of biotechnology and the biomanufacturing of recombinant proteins.

### BACKGROUND

Bacterial cells containing a nucleic acid that encodes a recombinant protein can be used to produce therapeutically or commercially important proteins. In the current environment of diverse product pipelines, biotechnology companies are increasingly driven to develop innovative solutions for highly flexible and cost-effective manufacturing of therapeutic protein drug substances.

### SUMMARY

Provided herein are processes for manufacturing recombinant ranibizumab or a ranibizumab variant. In some embodiments, a process as described herein includes: providing a liquid including recombinant ranibizumab or a ranibizumab variant that is substantially free of cells; capturing the recombinant ranibizumab or the ranibizumab variant in the liquid using an affinity chromatography column, where the eluate of the affinity chromatography column includes the recombinant ranibizumab or the ranibizumab variant; purifying the recombinant ranibizumab or the ranibizumab variant in the eluate from the affinity chromatography column using a first cation exchange chromatography column and buffers that have a pH of about pH 5.5 to about 7.5, wherein the eluate from the first cation exchange chromatography column includes the recombinant ranibizumab or the ranibizumab variant; and purifying the recombinant ranibizumab or the ranibizumab variant in the eluate from the first cation exchange chromatography column using a second cation chromatography column and buffers that have a pH of about pH 4.0 to about pH 5.4, where the eluate from the second cation chromatography column includes the recombinant ranibizumab or the ranibizumab variant.

In some embodiments, the liquid is a liquid culture medium that is substantially free of cells. In some embodiments, the liquid culture medium is a liquid culture medium harvested from a culture of bacteria that secrete ranibizumab or the ranibizumab variant. In some embodiments, the bacteria is selected from the group of: E. coli (e.g., K12 E. coli, BL21 E. coli, and 60E4 E. coli), Yersinia, Acinetobacter, Bordetlla, Brucella, Cyanobacter, Enterobacter, Helicobacter, Klebsiella, Neisseria, Pasteurella, Pseudomonas, Salmonella, and Shigella. In some embodiments, the culture of bacteria is a culture of E. coli that secrete recombinant ranibizumab or the ranibizumab variant. In some embodiments, the bacteria is a recombinant bacterium.

In some embodiments, the liquid culture medium has previously been subjected to acid precipitation.

Some embodiments further include performing acid precipitation on the liquid culture medium to provide a precipitate including contaminants and the liquid including the recombinant ranibizumab or the ranibizumab variant that is substantially free of cells (prior to the step of providing the liquid including recombinant ranibizumab or the ranibizumab variant that is substantially free of cells).

In some embodiments, the step of performing the acid precipitation on the liquid culture medium includes adjusting the pH of the liquid culture medium to about 3.5 to about 4.5. In some embodiments, the step of performing the acid precipitation on the liquid culture medium includes adjusting the pH of the liquid culture medium to about 3.8 to about 4.2. In some embodiments, the step of performing acid precipitation on the liquid culture medium includes the use of centrifugation.

Some embodiments further include filtering the liquid including the recombinant ranibizumab or the ranibizumab variant after providing the liquid including recombinant ranibizumab or the ranibizumab variant that is substantially free of cells and before capturing the recombinant ranibizumab or the ranibizumab variant in the liquid using an affinity chromatography column.

In some embodiments, the capturing is performed using an antibody- or antibody fragment-binding capture mechanism. In some embodiments, the antibody- or antibody-fragment binding capture mechanism is a kappa light chain-binding capture mechanism. In some embodiments, the affinity chromatography column includes a Capto Protein L chromatography resin.

In some embodiments, the step of purifying the recombinant ranibizumab or the ranibizumab variant in the eluate of the affinity chromatography column using the first cation exchange chromatography column is performed using buffered solutions having a pH of about 6.2 to about 6.8. In some embodiments, the step of purifying the recombinant ranibizumab or the ranibizumab variant in the eluate of the affinity chromatography column using the first cation exchange chromatography column is performed using buffered solutions having a pH of about 6.5.

In some embodiments, the first cation exchange chromatography column includes a CAPTO™ SP ImpRes chromatography resin.

Some embodiments further include adjusting ionic concentration of the eluate of the first cation exchange chromatography column after purifying the recombinant ranibizumab or the ranibizumab variant in the eluate of the affinity chromatography column using a first cation exchange chromatography column and before purifying the recombinant ranibizumab or the ranibizumab variant in the eluate from the first cation exchange chromatography column using a second cation chromatography column.

Some embodiments further include diluting the eluate of the first cation exchange chromatography column after purifying the recombinant ranibizumab or the ranibizumab variant in the eluate of the affinity chromatography column using a first cation exchange chromatography column and before purifying the recombinant ranibizumab or the ranibizumab variant in the eluate from the first cation exchange chromatography column using a second cation chromatography column.

In some embodiments, the step of purifying the recombinant ranibizumab or the ranibizumab variant in the eluate from the first cation exchange chromatography column using the second cation exchange chromatography column is performed using buffered solutions having a pH of about 4.5 to about 5.4. In some embodiments, the step of purifying the recombinant ranibizumab or the ranibizumab variant in the eluate from the first cation exchange chromatography column using the second cation exchange chromatography column is performed using buffered solutions having a pH of about 4.8 to about 5.2. In some embodiments, the step of purifying the recombinant ranibizumab or the ranibizumab variant in the eluate from the first cation exchange chromatography column using the second cation exchange chromatography column is performed using buffered solutions having a pH of about 5.0.

In some embodiments, the second cation exchange chromatography column includes a CAPTO™ SP ImpRes chromatography resin.

Some embodiments further include filtering the eluate from the second cation exchange chromatography column after purifying the recombinant ranibizumab or the ranibizumab variant in the eluate from the first cation exchange chromatography column using a second cation chromatography column.

Some embodiments further include adjusting ionic concentration of the eluate of the second cation exchange chromatography column after purifying the recombinant ranibizumab or the ranibizumab variant in the eluate from the first cation exchange chromatography column using a second cation chromatography column.

Some embodiments further include diluting the eluate of the second cation exchange chromatography column after purifying the recombinant ranibizumab or the ranibizumab variant in the eluate from the first cation exchange chromatography column using a second cation chromatography column.

Some embodiments further include purifying and polishing the recombinant ranibizumab or the ranibizumab variant in the eluate from the second cation exchange chromatography column using a hydrophobic interaction chromatography column, wherein the eluate of the hydrophobic interaction chromatography column includes the recombinant ranibizumab or the ranibizumab variant, after purifying the recombinant ranibizumab or the ranibizumab variant in the eluate from the first cation exchange chromatography column using a second cation chromatography column. In some embodiments, the hydrophobic interaction chromatography column includes a phenyl sepharose chromatography resin. In some embodiments, the process further includes filtering the eluate from the hydrophobic interaction chromatography column.

Also provided herein are pharmaceutical compositions that include a therapeutically effective amount of ranibizumab or a ranibizumab variant produced by any of the methods described herein. In some embodiments of any of the pharmaceutical compositions described herein, the pharmaceutical composition includes about 5 mg/mL to about 15 mg/mL ranibizumab or a ranibizumab variant. In some embodiments of any of the pharmaceutical compositions described herein, the pharmaceutical composition includes about 10 mg/mL ranibizumab or a ranibizumab variant. Some embodiments of any of the pharmaceutical compositions described herein further include a tonicity agent, a buffer, a surfactant, and water for injection, and where the pharmaceutical composition has a pH of about 5 to about 6. In some embodiments of any of the pharmaceutical compositions described herein, the pharmaceutical composition includes trehalose, a histidine buffer, a polysorbate, and water for injection, and the pharmaceutical composition has a pH of about 5 to about 6. In some embodiments, the pharmaceutical composition includes α,α-trehalose dihydrate, histidine HCl, polysorbate 20, and water for injection, and the pharmaceutical composition has a pH of about 5 to about 6. In some embodiments the pH of the composition is about 5.5. In some embodiments, the pharmaceutical composition includes 6 mg/mL ranibizumab or a ranibizumab variant. In some embodiments, the pharmaceutical composition includes 6 mg/mL ranibizumab or a ranibizumab variant in 0.05 mL (0.3 mg dose prefilled syringe or vial). In some embodiments, the pharmaceutical composition includes 10 mg/mL ranibizumab or a ranibizumab variant. In some embodiments, the pharmaceutical composition includes 10 mg/mL ranibizumab or a ranibizumab variant in 0.05 mL (0.5 mg dose prefilled syringe or vial).

Also provided herein are kits that include any of the pharmaceutical compositions described herein. Some embodiments of any of the kits described herein further include a sterile glass vial, where the pharmaceutical composition is disposed within the sterile glass vial. Some embodiments of any of the kits described herein further include a syringe, where the pharmaceutical compositions is disposed within the syringe. In some embodiments, the pharmaceutical composition is supplied as a preservative-free, sterile solution in a single-use container designed to deliver 0.05 mL of 10 mg/mL ranibizumab or a ranibizumab variant (0.5 mg dose prefilled syringe or vial) or 6 mg/mL ranibizumab or a ranibizumab variant (0.3 mg dose prefilled syringe or vial) aqueous solution with 10 mM histidine HCl, 10% α,α-trehalose dihydrate, 0.01% polysorbate 20, pH 5.5.

Also provided herein are methods of treating a subject in need thereof that include administering to the subject a therapeutically effective amount of any of the pharmaceutical compositions described herein. In some embodiments of any of the methods of treating a subject described herein, the subject has been identified or diagnosed as having wet age-related macular degeneration, diabetic macular edema, or macular edema following retinal vein occlusion (e.g., branch retinal vein occlusion or central retinal vein occlusion).

As used herein, the word “a” before a noun represents one or more of the particular noun. For example the “a buffer” represents “one or more buffers.”

The term “bacterium” or “bacterial cell” means any cell from or derived from any bacterium (e.g., a Gram positive bacterium, or a Gram negative bacterium). Non-limiting examples of bacteria are described herein. Additional examples of bacteria are known in the art.

The term “recombinant bacterium” means a bacterium that contains a nucleic acid that is not naturally present in the bacterium. For example, the nucleic acid that is not naturally present in the bacterium can encode a recombinant protein (e.g., ranibizumab or a ranibizumab variant) and/or can encode a selectable marker (e.g., any of the exemplary selectable markers described herein). The nucleic acid that is not naturally present in the cell can, e.g., be integrated into the genome of the bacterium. In other examples, the nucleic acid that is not naturally present in the cell is not integrated into the genome of the bacterium. For example, a nucleic acid that is not naturally present in the bacterium can be episomal.

The term “substantially free” means a composition (e.g., a liquid culture medium) that is at least or about 90% free (e.g., at least or about 95%, 96%, 97%, 98%, or at least or about 99% free, or about 100% free) of a specified substance (e.g., a mammalian cell).

The term “culturing” or “cell culturing,” as used herein, refers to the maintenance or proliferation of a bacterium (e.g., a recombinant bacterium) under a controlled set of physical conditions.

The term “liquid culture medium” comprises a fluid that contains sufficient nutrients to allow a cell (e.g., a bacterial cell) to grow or proliferate in vitro. In some examples, a liquid culture medium can include a recombinant protein produced by a recombinant bacterium. In some embodiments, a liquid culture medium is a minimal medium (e.g., a medium containing only inorganic salts, a carbon source, and water). Non-limiting examples of a liquid culture medium are described herein. Additional examples of a liquid culture medium are described in the art and are commercially available. In some examples, a liquid culture medium can be intended for addition, as a feed liquid culture medium, to an already prepared recombinant protein production medium (as defined below).

The term “immunoglobulin” means a polypeptide containing an amino acid sequence of at least 15 amino acids (e.g., at least 20, 30, 40, 50, 60, 70, 80, 90, or 100 amino acids) of an immunoglobulin protein (e.g., a variable domain sequence, a framework sequence, or a constant domain sequence). The immunoglobulin may, for example, include at least 15 amino acids of a light chain immunoglobulin, e.g., at least 15 amino acids of a heavy chain immunoglobulin. The immunoglobulin may be an isolated antibody (e.g., an IgG, IgE, IgD, IgA, or IgM). The immunoglobulin may be a subclass of IgG (e.g., IgG1, IgG2, IgG3, or IgG4). The immunoglobulin may be an antibody fragment, e.g., a Fab fragment, a F(ab′)2 fragment, or an a scFv fragment. The immunoglobulin may also be a bi-specific antibody or a tri-specific antibody, or a dimer, trimer, or multimer antibody, or a diabody, an Affibody®, or a Nanobody®. The immunoglobulin can also be an engineered protein containing at least one immunoglobulin domain (e.g., a fusion protein). Non-limiting examples of immunoglobulins are described herein and additional examples of immunoglobulins are known in the art.

The term “protein fragment” or “polypeptide fragment” means a portion of a polypeptide sequence that is at least or about 4 amino acids, at least or about 5 amino acids, at least or about 6 amino acids, at least or about 7 amino acids, at least or about 8 amino acids, at least or about 9 amino acids, at least or about 10 amino acids, at least or about 11 amino acids, at least or about 12 amino acids, at least or about 13 amino acids, at least or about 14 amino acids, at least or about 15 amino acids, at least or about 16 amino acids, at least or about 17 amino acids, at least or about 18 amino acids, at least or about 19 amino acids, or at least or about 20 amino acids in length, or more than 20 amino acids in length. A recombinant protein fragment can be produced using any of the processes described herein.

The term “unit operation” is a term of art and means a functional step that can be performed in a process of manufacturing a recombinant protein drug substance from a liquid culture medium. For example, a unit of operation can be filtering (e.g., removal of contaminant bacteria, yeast viruses, or mycobacteria, and/or particular matter from a fluid containing a recombinant therapeutic protein), capturing, epitope tag removal, purifying, holding or storing, polishing, viral inactivating, adjusting the ionic concentration and/or pH of a fluid containing the recombinant protein, and removing unwanted salts.

The term “capturing” means a step performed to partially purify or isolate (e.g., at least or about 5%, e.g., at least or about 10%, 15%, 20%, 25%, 30%, 40%, 45%, 50%, 55%, 60%, 65%, 70%, 75%, 80%, 85%, 90%, or at least or about 95% pure by weight), concentrate, and stabilize a recombinant protein from one or more other components present in a liquid culture medium or a diluted liquid culture medium (e.g., culture medium proteins or one or more other components (e.g., DNA, RNA, or other proteins) present in or secreted from a mammalian cell). Typically, capturing is performed using a resin that binds a recombinant protein (e.g., through the use of affinity chromatography). Non-limiting methods for capturing a recombinant protein from a liquid culture medium or diluted liquid culture medium are described herein and others are known in the art. A recombinant protein can be captured from a liquid culture medium using at least one chromatography column and/or chromatographic membrane (e.g., any of the chromatography columns and/or chromatographic membranes described herein).

The term “recovering,” as used herein, comprises at least partially purifying or isolating (e.g., at least or about 4% (e.g., at least or about 5%, 10%, 12%, 15%, 20%, 25%, 30%, 35%, 40%, 45%, 50%, 55%, 60%, 65%, 70%, 75%, 80%, 85%, 90% or 95%) a recombinant protein from one or more components present in a liquid culture medium (e.g., recombinant bacterial cells, or one or more other components (e.g., DNA, RNA, or other proteins) present in a recombinant bacterial cell lysate). Non-limiting methods for recovering a recombinant protein from a liquid culture medium or from a recombinant bacterial cell lysate are described herein and are disclosed in the art.

The term “purifying” means a step performed to isolate a recombinant protein from one or more other impurities (e.g., bulk impurities) or components present in a fluid containing a recombinant protein (e.g., liquid culture medium proteins or one or more other components (e.g., DNA, RNA, other proteins, endotoxins, viruses, etc.) present in or secreted from a bacterial cell). For example, purifying can be performed during or after an initial capturing step. Purification can be performed using a resin, membrane, or any other solid support that binds either a recombinant protein or contaminants (e.g., through the use of affinity chromatography, hydrophobic interaction chromatography, anion or cation exchange chromatography, or molecular sieve chromatography). A recombinant protein can be purified from a fluid containing the recombinant protein using at least one chromatography column and/or chromatographic membrane (e.g., any of the chromatography columns or chromatographic membranes described herein).

The term “polishing” is a term of art and means a step performed to remove remaining trace or small amounts of contaminants or impurities from a fluid containing a recombinant protein that is close to a final desired purity. For example, polishing can be performed by passing a fluid containing the recombinant protein through a chromatographic column(s) or membrane absorber(s) that selectively binds to either the target recombinant protein or small amounts of contaminants or impurities present in a fluid containing a recombinant protein. In such an example, the eluate/filtrate of the chromatographic column(s) or membrane absorber(s) contains the recombinant protein.

The term “filtering” means the removal of at least part of (e.g., at least 80%, 90%, 95%, 96%, 97%, 98%, or 99%) undesired biological contaminants (e.g., a mammalian cell, bacteria, yeast cells, viruses, or mycobacteria) and/or particulate matter (e.g., precipitated proteins) from a liquid (e.g., a liquid culture medium or fluid present in any of the systems or processes described herein).

The term “secreted protein” or “released protein,” as used herein comprises a protein (e.g., a recombinant protein) that originally contained at least one secretion signal sequence when it is translated within a bacterial cell, and through, at least in part, enzymatic cleavage of the secretion signal sequence in the bacterial cell, is secreted into the extracellular space. In some embodiments, the extracellular space is a liquid culture medium. Skilled practitioners will appreciate that a “secreted” protein need not dissociate entirely from the bacterium outer membrane to be considered a secreted protein. As used herein, a secreted recombinant protein does not refer to a protein retained in the periplasm of a recombinant bacterium.

The term “fed-batch bioreactor,” as used herein, refers to a bioreactor containing, or suitable for containing, a plurality of bacteria (e.g., recombinant bacteria) in a starting volume of a liquid culture medium, where the cell culture present in the bioreactor includes the periodic or continuous addition of an additional volume of a feed liquid culture medium to the starting volume of the liquid culture medium, without substantial or significant removal of liquid culture medium from the cell culture. In some embodiments, the feed liquid culture medium added is the same as the starting volume of the liquid culture medium. In some examples of fed-batch culture, the later-added feed liquid culture medium is a concentrated form of the starting volume of the liquid culture medium.

The term “yield” refers to a ratio of a total amount of recombinant protein obtained over a theoretical amount of recombinant protein that could be obtained (expressed as a percentage), based on the amount of starting materials.

The term “promoter” is a nucleic acid sequence that is operably linked to a nucleic acid sequence encoding a protein (e.g., a recombinant protein) that can increase the transcription of the nucleic acid sequence encoding the protein. In some aspects, a promoter is constitutive. In other aspects, a promoter is inducible. Non-limiting examples of promoters are described herein. Additional examples of promoters are known in the art.

The term “therapeutic protein drug substance” means a recombinant protein (e.g., ranibizumab) that has been sufficiently purified or isolated from contaminating proteins, lipids, and nucleic acids (e.g., contaminating proteins, lipids, and nucleic acids present in a liquid culture medium or from a host cell (e.g., from a mammalian, yeast, or bacterial host cell)) and biological contaminants (e.g., viral and bacterial contaminants), and can be formulated into a pharmaceutical agent without any further substantial purification and/or decontamination step.

The term “formulating,” as used herein, comprises processing a recombinant protein (e.g., ranibizumab or a ranibizumab variant) in a format that is compatible with its intended route of administration (e.g., intraarterial, intradermal, intramuscular, intravenous, intraperitoneal, subcutaneous, or oral) to a subject. In some aspects, formulating results in the provision of the recombinant protein (e.g., ranibizumab or a ranibizumab variant) in a dosage unit form (i.e., physically discrete units containing a predetermined quantity of recombinant ranibizumab or a ranibizumab variant for ease of administration and uniformity of dosage). In some aspects, formulating can include a sterile diluent (e.g., sterile water, saline, or water for injection), a fixed oil, polyethylene glycol, glycerin, propylene glycol, or other synthetic solvents, antibacterial or antifungal agents (e.g., benzyl alcohol, methyl parabens, chlorobutanol, phenol, ascorbic acid, thimerosal), antioxidants (e.g., ascorbic acid or sodium bisulfite), chelating agents (e.g., ethylenediaminetetraacetic acid), buffers (e.g., acetates, citrates, phosphates), isotonic agents (e.g., sugars, polyalcohols (e.g. mannitol or sorbitol), or salts (e.g., sodium chloride), or any combination thereof. Non-limiting examples of formulations of recombinant ranibizumab or a ranibizumab variant are described herein.

Unless otherwise defined, all technical and scientific terms used herein have the same meanings as commonly understood by one of ordinary skill in the art to which this disclosure belongs. Methods and materials are described herein for use in the present disclosure; other, suitable methods and materials described in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIG. 1 is an exemplary schematic showing steps to purify ranibizumab.

FIG. 2 is a graph of the real-time UV profile of the CAPTO™ L capture column over a limited time window. Shown is the absorbance at 280 nm, the absorbance at 300 nm, and the conductivity.

FIG. 3 is a graph of the real-time UV profile of the CAPTO™ SP ImpRes ion exchange column at pH 6.5 over a limited time window. Shown is the UV absorbance at 280 nm, the concentration of buffer B (%) as described in Example 1, and the conductivity (mS/cm).

FIG. 4 is a graph of the real-time UV profile of the CAPTO™ SP ImpRes ion exchange column at pH 5.0 over a limited time window. Shown is the UV absorbance at 280 nm, the conductivity, and the event markers.

FIG. 5 is a graph of the real-time UV profile of the phenyl column over a limited time window. Shown is the UV absorbance at 280 nm, the conductivity, and the shaded area shows pooling area.

FIG. 6 is a graph of the real-time UV elution profile of a CAPTO™ SP ImpRes ion exchange column at pH 6.5 of an exemplary lot of ranibizumab (Lot #2) over a limited time window. Shown is the UV absorbance at 280 nm, the concentration of buffer B (%), and the conductivity (mS/cm).

FIG. 7 is a graph of the real-time UV profile UV profile of the CAPTO™ SP ImpRes ion exchange column at pH 5.0 of an exemplary lot of ranibizumab (Lot #2) over a limited time window. Shown is the UV absorbance at 280 nm and the % B buffer.

FIG. 8 is a graph of the real-time UV profile of the phenyl column of an exemplary lot of ranibizumab (Lot #2) over a limited time window. Shown is the UV absorbance at 280 nm and the red trace is conductivity.

FIGS. 9A and 9B are chromatograms of a CEX-HPLC assay comparing samples at different stages of the purification process that used Lot #2 as the starting material. Shown is an elution sample from column 1, an elution sample from column 2, an elution sample from column 3, an elution sample from column 4, and the sample after UF/DF using a posidyne filter. FIG. 9B is a magnified image of FIG. 9A.

FIGS. 10A and 10B are chromatograms of a CEX-HPLC assay comparing different lots of ranibizumab (Lot #2, Lot #3, and Lucentis). FIG. 10B is a magnified image of FIG. 10A.

FIGS. 11A and 11B are chromatograms of an SEC-HPLC assay comparing samples at different stage of the purification process that used Lot #2 as the starting material. Shown is an elution sample from column 1, an elution sample from column 2, an elution sample from column 3, an elution sample from column 4, and the final sample after UF/DF performed using a posidyne filter. FIG. 11B is a magnified version of FIG. 11A.

FIGS. 12A and 12B are chromatograms of a CEX-HPLC assay comparing different lots of ranibizumab (Lot #2, Lot #3, and Lucentis). FIG. 12B is a magnified image of FIG. 12A.

FIGS. 13A and 13B are chromatograms of an nrRP-HPLC assay comparing samples at different stages of the purification process that used Lot #2 as the starting material. FIG. 13B is a magnified image of FIG. 13A.

FIGS. 14A and 14B are chromatograms of an nrRP-HPLC assay comparing different lots of ranibizumab (Lot #2, Lot #3, and Lucentis). FIG. 14B is a magnified image of 14A.

### DETAILED DESCRIPTION

Provided herein are processes for manufacturing recombinant ranibizumab or a ranibizumab variant. In some embodiments, a process as described herein includes: providing a liquid including recombinant ranibizumab or a ranibizumab variant that is substantially free of cells; capturing the recombinant ranibizumab or ranibizumab variant in the liquid using an affinity chromatography column, where the eluate of the affinity chromatography column includes the recombinant ranibizumab or ranibizumab variant; purifying the recombinant ranibizumab or ranibizumab variant in the eluate from the affinity chromatography column using a first cation exchange chromatography column and buffers that have a pH of about pH 5.5 to about 7.5, where the eluate from the first cation exchange chromatography column includes the recombinant ranibizumab or ranibizumab variant; and purifying the recombinant ranibizumab or ranibizumab variant in the eluate from the first cation exchange chromatography column using a second cation chromatography column and buffers that have a pH of about pH 4.0 to about pH 5.4, where the eluate from the second cation chromatography column includes the recombinant ranibizumab or ranibizumab variant.

### Ranibizumab and Ranibizumab Variants

Ranibizumab is a recombinant humanized IgG1 kappa isotype monoclonal antibody fragment. Ranibizumab binds to and inhibits the biologic activity of human vascular endothelial growth factor A (VEGF-A). Ranibizumab, which lacks an Fc region, has a molecular weight of approximately 48 kilodaltons. Ranibizumab binds to the receptor binding site of active forms of VEGF-A, including the biologically active, cleaved form of this molecule, VEGF110. The binding of ranibizumab to VEGF-A prevents the interaction of VEGF-A with its receptors (VEGFR1 and VEGFR2) on the surface of endothelial cells, reducing endothelial cell proliferation, vascular leakage, and new blood vessel formation. Ranibizumab has been described in U.S. Pat. No. 6,407,213 (in addition to non-human CDRs derived from the sequence of the murine antibody, ranibizumab comprises framework substitutions in the variable domains at positions 4 and 46 in the light chain (VL) and positions 49, 69, 71, 73, 76, 78, and 94 in the heavy chain (VH)), and in U.S. Pat. No. 7,060,269 (Y0317 light chain SEQ ID NO: 1 and heavy chain SEQ ID NO: 2).

Light Chain
(SEQ ID NO: 1)
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTV
Heavy Chain
(SEQ ID NO: 2)
SGGGSGSGDFDYEKMANANKGAMTENADENALQSDAKGKLDSVATDYGAA
IDGFIGDVSGLANGNGATGDFAGSNSQMAQVGDGDNSPLMNNFRQYLPSL
PQSVECRPFVFSAGKPYEFSIDCDKINLFRGVFAFLLYVATFMYVFSTFA
NILRNKES

In some embodiments, ranibizumab includes the amino acid sequence of one or both of SEQ ID NO: 3 and 4.

Light Chain of Ranibizumab
(SEQ ID NO: 3)
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Heavy Chain of Ranibizumab
(SEQ ID NO: 4)
EVQLVESGGGLVQPGGSLRLSCAASGYDFTHYGMETNWVRQAPGKGLEWV
GWINTYTGEPTYAADFKRRFTFSLDTSKSTAYLQMETNSLRAEDTAVYYC
AKYPYYYGTSHWYFDVWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHL

Any of the methods described herein can also be performed using a ranibizumab variant. A ranibizumab variant, e.g., can have 1 to 25 (e.g., 1 to 20, 1 to 15, 1 to 10, one to 10, 1 to 8, 1 to 6, 1 to 4, 1 to 3, or 1 or 2 amino acid substitutions in either the heavy chain (e.g, SEQ ID NO: 4) and/or the light chain (e.g., SEQ ID NO: 3) of ranibizumab.

In some embodiments, a ranibizumab variant can include an amino acid sequence that is at least 80% (e.g., at least 81%, at least 82%, at least 83%, at least 84%, at least 85%, at least 86%, at least 88%, at least 90%, at least 92%, at least 94%, at least 95%, at least 96%, at least 97%, at least 98%, at least 99%, at least 99.5%, or 100%) identical to SEQ ID NO: 3. In some embodiments, a ranibizumab variant can include an amino acid sequence that is at least 80% (e.g., at least 81%, at least 82%, at least 83%, at least 84%, at least 85%, at least 86%, at least 88%, at least 90%, at least 92%, at least 94%, at least 95%, at least 96%, at least 97%, at least 98%, at least 99%, at least 99.5%, or 100%) identical to SEQ ID NO: 4.

In some embodiments, a variant ranibizumab can include a sequence that differs from the amino sequence of SEQ ID NO: 3 by 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) amino acids. In some embodiments, a variant ranibizumab can include a sequence that differs from the amino sequence of SEQ ID NO: 4 by 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) amino acids.

For example, where a variant ranibizumab includes a sequence that differs from the amino acid sequence of SEQ ID NO: 3 and/or SEQ ID NO: 4 by one or more amino acids, the amino acid present in SEQ ID NO: 3 and/or SEQ ID NO: 4 can be replaced by a similar amino acid. For example, a serine can be replaced by any of glycine, alanine, serine, threonine, or proline; arginine can be replaced by asparagine, lysine, glutamine, or histidine; leucine can be replaced by phenylalanine, isoleucine, valine, or methionine; proline can be replaced with glycine, alanine, serine, or threonine; alanine can be replaced with glycine, threonine, proline, or serine; valine can be replaced with methionine, phenylalanine, isoleucine, or leucine; glycine can be replaced with alanine, threonine, proline, or serine; isoleucine can be replaced with phenylalanine, valine, leucine, or methionine; phenylalanine can be replaced with tryptophan or tyrosine; tyrosine can be replaced with tryptophan or phenylalanine; cysteine can be replaced with serine or threonine; histidine can be replaced with asparagine, lysine, glutamine, or arginine; glutamine can be replaced with glutamic acid, asparagine, or aspartic acid; asparagine can be replaced with glutamic acid, aspartic acid, or glutamine; lysine can be replaced with asparagine, glutamine, arginine, or histidine; asparatic acid can be replaced with glutamic acid, asparagine, or glutamine; glutamic acid can be replaced by asparagine, aspartic acid, or glutamine; methionine can be replaced with phenylalanine, isoleucine, valine, or leucine; and tryptophan can be replaced with phenylalanine or tyrosine.

In some examples, a precursor form of a recombinant protein (e.g., ranibizumab or a ranibizumab variant) can include a signal sequence. Non-limiting examples of signal sequences include:

(SEQ ID NO: 5)
MKYLLPTAAAGLLLLAAQPAMA;
(SEQ ID NO: 6)
MKKTAIAIAVALAGFATVAQA;
(SEQ ID NO: 7)
MKKNIAFLLASMFVFSIATNAYA;
(SEQ ID NO: 8)
MFKFKKKFLVGLTAAFMSISMFSATASA;
(SEQ ID NO: 9)
MKQSTAILALLPLLFTPVTKA;
(SEQ ID NO: 10)
MMKRNILAVIVPALLVAGTANA;
(SEQ ID NO: 11)
MKKSTLALVVMGIVASASVQA;
(SEQ ID NO: 12)
MKIKTGARILALSALTTMMFSASALA;
(SEQ ID NO: 13)
MKVKVLSLLVPALLVAGAANA;
(SEQ ID NO: 14)
MKATKLVLGAVILGSTLLAG;
(SEQ ID NO: 15)
MMITLRKLPLAVAVAAGVMSAQAMA;
(SEQ ID NO: 16)
MRAKLLGIVLTTPIAISSFA;
(SEQ ID NO: 17)
MSIQHFRVALIPFFAAFCLPVFA;
(SEQ ID NO: 18)
MKQSTIALALLPLLFTPVTKA;
(SEQ ID NO: 19)
MKVMRTTVATVVAATLSMSAFSVFA;
and
(SEQ ID NO: 20)
MKKSLVLKASVAVATLVPMLSFA.

A recombinant protein (e.g., ranibizumab or a ranibizumab variant) can be recovered and purified from a liquid (e.g., a liquid culture medium, a diluted liquid culture medium, or a processed liquid culture medium) using any of methods described herein.

In some embodiments, at least about 30% (e.g., at least about 40%, at least about 50%, at least about 55%, at least about 60%, at least about 65%, at least about 70%, at least about 75%, at least about 80%, at least about 85%, at least about 90%, at least about 95%, or at least about 99%) of a recombinant protein (e.g., ranibizumab or a ranibizumab variant) is properly folded or unfolded in the liquid culture medium.

In some embodiments, less than about 30% (e.g., less than about 25%, less than about 20%, less than about 15%, less than about 10%, less than about 8%, less than about 6%, less than about 4%, less than about 2%, or less than about 1%) of the recombinant protein (e.g., ranibizumab or a ranibizumab variant) is not properly folded or unfolded in the liquid culture medium.

### Recombinant Bacterium

In some embodiments, ranibizumab or a ranibizumab variant is expressed by a recombinant bacterium and secreted into a liquid culture medium. For example, in some embodiments, the recombinant bacterium is a recombinant bacterium (e.g., a Gram-negative bacterium) including a nucleic acid encoding ranibizumab or a ranibizumab variant. In some embodiments of any of the recombinant bacteria described herein, the recombinant bacterium can have a type II secretion mechanism (described in, e.g., von Tils et al., Front. Cell Infect. Microbiol. 2(160):1-11, 2012, and Mergulhao et al., Biotechnol. Advances 23:177-202, 2005).

Non-limiting examples of a recombinant bacterium that can be used to express a recombinant protein (e.g., ranibizumab) include: an E. coli strain (e.g., a BL21 E. coli strain, a 60E4 E. coli strain, E. coli JM109 strain, E. coli MC4100 strain, E. coli DH5α strain, E. coli KS476 (degP) strain, E. coli TX1 strain, E. coli HB101 strain, E. coli JM105 strain, E. coli TOP10 strain, E. coli TOP10 F′ strain, E. coli 60E4 strain, E. coli RV308 strain, E. coli BL21 (DE3) strain, E. coli HM114 strain, E. coli TB1, E. coli K12 strain, E. coli AF1000 strain, E. coli MC1061 strain, E. coli CC118 strain, E. coli RB791 strain, E. coli CC202 strain, E. coli SF110 strain, E. coli TG1 strain, E. coli HB2151 strain, E. coli XL1-Blue MRF′ strain, E. coli WCM105 strain, or E. coli C600-1 strain), a Yersinia strain, an Acinetobacter strain, a Bordella strain, a Brucella strain, a Cyanobacter strain, an Enterobacter strain, a Helicobacter strain, a Klebsiella strain, a Neisseria strain, a Pasteurella strain, a Pseudomonas strain (e.g., Pseudomonas arginosa, Pseudomonas fluorescens), a Salmonella strain, Ralstonia strain (e.g., Ralstonia eutropha), and a Shigella strain.

In some embodiments, the recombinant bacterium includes at least one nucleic acid that encodes SEQ ID NO: 3. In some examples, the recombinant bacterium includes at least one nucleic acid that encodes SEQ ID NO: 4. In some examples, the recombinant bacterium includes one nucleic acid that encodes SEQ ID NO: 3 and SEQ ID NO: 4. In some examples, the recombinant bacterium includes a first nucleic acid encoding SEQ ID NO: 3 and a second nucleic acid encoding SEQ ID NO: 4.

In some embodiments, the recombinant bacterium expresses a nucleic acid that is under control of at least one promoter (e.g., operably linked to at least one promoter). Non-limiting examples of promoters include a phoA promoter, a lac promoter, a tac promoter, a phoA promoter, a tetA promoter, a araBAD promoter, a T7 promoter, a T7/lac promoter, a lacUV5 promoter, a lacUT75 promoter, a trc promoter, or a cspA promoter. Additional promoters that can be used to express a nucleic acid in a recombinant bacterium are known in the art.

Non-limiting examples of expression vectors that can be used to express ranibizumab include pOPE101-XP, pTTO-1, or pDNAbEng-1. In some examples of these expression vectors, the expression can include an origin of replication. Non-limiting examples of origins of replication include pMB1, ColE1, pUC, p15A, or pSC101.

In some examples of these expression vectors, the expression vector can further include a selectable marker, e.g., an antibiotic resistance gene (e.g., Amp, Cm, Tet, or Kan resistance gene). Additional examples of expression vectors and origins of replication and promoters that can be used in expression vectors are described in U.S. Pat. No. 5,595,898, U.S. Patent Application Publication No. 2012/0137162, U.S. Pat. No. 7,754,447, U.S. Reissued Pat. No. 44,512, European Patent No. 1664278, and U.S. Pat. No. 9,267,164 (incorporated herein by reference).

### Liquid Culture Medium and Cell Culturing

Liquid culture media include ingredients and nutrients that are generally useful for bacterial growth (e.g., a carbon source (e.g., glucose, glutamate, galactose, citric acid, glycerol, or a combination thereof) and a nitrogen source (e.g., yeast extract, ammonium salt, nitrates)). In some embodiments, liquid culture media are supplemented with an antibiotic (e.g., tetracycline), and/or a phosphate (e.g., inorganic phosphate). In some embodiments, the liquid culture media include animal-free components. In some embodiments, the liquid culture media can include tryptone and/or N-Z amine type A.

Cell culturing includes incubating a recombinant bacteria (e.g., any of the recombinant bacteria described herein) in a liquid culture medium under a controlled set of conditions. In some embodiments, cell culturing includes a proliferation phase (i.e., cell mass accumulation and rapid cell growth) and a production phase (i.e., recombinant protein production phase). In some aspects, the production phase is initiated upon the depletion of a component of the cell culture medium (e.g., depletion of phosphate).

In some embodiments, the culturing is fed batch culturing. In some embodiments, the fed batch culturing includes continuously or periodically adding a feed liquid culture medium to a starting volume of a liquid culture medium. In some embodiments, the fed batch culturing includes continuously adding to the starting volume of a liquid culture medium, an additional volume of a feed liquid culture medium. In some embodiments, the fed batch culturing includes periodically adding to a starting volume of a liquid culture medium, an additional volume of a feed liquid culture medium. In a non-limiting example, periodically adding a feed liquid culture medium includes adding a volume of a feed liquid culture medium once every 6, 12, or 24 hours.

In some embodiments, the culturing is performed using a shake flask. In some embodiments, the culturing is performed using a bioreactor or fermentor. In some embodiments, the culturing is performed using a fed batch bioreactor or fermentor.

### Recovering Recombinant Protein

In some embodiments, a recombinant protein (e.g., ranibizumab or a ranibizumab variant) is harvested from a liquid culture medium by removing, or otherwise physically separating the liquid culture medium from the recombinant bacteria. A variety of different methods for removing liquid culture medium from recombinant bacteria are known in the art, including, for example, centrifugation, filtration, pipetting, and/or aspiration. In some embodiments of any of the methods described herein, the method does not include a physical (e.g., homogenization, French press, glass or ceramic beads, sonication, autoclave, microwave, freeze/thawing, or mortar and pestle) or a chemical step (e.g., enzymes, e.g., such as cellulases, chitinases, lysozyme, mannase, or glycanase; an alcohol; chloroform; ether; or a chelator (e.g., EDTA)) to disrupt the outer membrane of the recombinant bacteria.

### Purifying a Recombinant Protein (e.g., Ranibizumab or a Ranibizumab Variant)

In some embodiments, a recombinant protein (e.g., ranibizumab or a ranibizumab variant) is purified from a liquid, e.g., a liquid culture medium (e.g., a liquid culture medium that is substantially free of recombinant bacteria), a diluted liquid culture medium (e.g., a diluted liquid culture medium that is substantially free of recombinant bacteria), or a processed liquid culture medium (e.g., a liquid culture medium that is substantially free of recombinant bacteria and has been subjected to acid precipitation and/or clarification) using any of the methods described herein. In some examples, purifying a recombinant protein (e.g., ranibizumab or a ranibizumab variant) includes the performance of one or more of: filtration, capturing, purification, and polishing.

Acid Precipitation

In some embodiments, the method of purifying recombinant protein (e.g., ranibizumab or a ranibizumab variant) can include, at least in part, a step of subjecting a liquid including recombinant protein (e.g., ranibizumab or a ranibizumab variant) (e.g., a liquid culture medium, e.g., a liquid culture medium that is substantially free of recombinant bacteria) to acid precipitation. Acid precipitation can provide a precipitate comprising contaminants and a liquid including the recombinant ranibizumab or ranibizumab variant. In some embodiments, subjecting a liquid including recombinant ranibizumab or ranibizumab variant (e.g., a culture medium including ranibizumab or a ranibizumab variant that is substantially free of recombinant bacteria) to acid precipitation includes adjusting the pH of the recombinant protein production medium to a pH of about 3 to about 4.5 (e.g., about 3 to about 3.4, about 3 to about 3.8, about 3 to about 4.2, about 3 to about 4.5, about 3.8 to about 4.5, about 3.4 to about 4.5, about 3.4 to about 3.8, about 3.4 to about 4.2, about 3.4 to about 4.2, about 3.8 to about 4.2, or about 3.8 to about 4.5). In some embodiments, subjecting a liquid including recombinant ranibizumab or ranibizumab variant (e.g., a liquid culture medium including recombinant ranibizumab or ranibizumab variant, and optionally, substantially free of recombinant bacteria) to acid precipitation includes adjusting the pH of the liquid including recombinant ranibizumab or ranibizumab variant to a pH of about 3, about 3.1, about 3.2, about 3.3, about 3.4, about 3.5, about 3.6, about 3.7, about 3.8, about 3.9, about 4, about 4.1, about 4.2, about 4.3, about 4.4, or about 4.5.

In some embodiments, a strong acid or a solution comprising a strong acid is added to a liquid including the recombinant protein (e.g., ranibizumab or a ranibizumab variant) (e.g., a liquid culture medium including recombinant ranibizumab or ranibizumab variant, and optionally, substantially free of recombinant bacteria). Non-limiting examples of strong acids include HCl, HI, HBr, HClO₄, HClO₃, HNO₃, and H₂SO₄. In some embodiments, the acid (e.g., a strong acid or a solution comprising a strong acid) is added with continues mixing.

In some embodiments, after the acid is added to the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria), the liquid including the recombinant protein (e.g., ranibizumab or a ranibizumab variant) is incubated for a period of time, e.g., a period of time of about 1 hour to about 5 hours, about 1.5 hours to about 5 hours, about 2 hours to about 5 hours, about 2.5 hours to about 5 hours, about 3 hours to about 5 hours, about 3.5 hours to about 5 hours, about 4 hours to about 5 hours, about 4.5 to about 5 hours, about 4 hours to about 5 hours, about 3.5 hours to about 5 hours, about 3 hours to about 5 hours, about 2.5 about to about 5 hours, about 2 hours to about 5 hours, about 1.5 hours to about 5 hours, about 2 hours to about 5 hours, about 3 hours to about 5 hours, about 3 hours to about 4 hours, about 2 hours to about 4 hours, or about 1 hour to about 3 hours. In some embodiments, the recombinant protein production medium is incubated for a period of time of about 1 hour, about 1.5 hours, about 2 hours, about 2.5 hours, about 3 hours, about 3.5 hours, about 4 hours, about 4.5 hours, or about hours, e.g., at a temperature of about 4° C. to about 25° C., about 4° C. to about 20° C., about 4° C. to about 15° C., about 4° C. to about 10° C., about 6° C. to about 25° C., about 6° C. to about 20° C., about 6° C. to about 15° C., about 6° C. to about 10° C., about 10° C. to about 25° C., about 10° C. to about 20° C., about 10° C. to about 15° C., about 15° C. to about 25° C., about 15° C. to about 20° C., or about 20° C. to about 25° C.

In some embodiments, the step of performing acid precipitation on the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria) further comprises the use of centrifugation. In some embodiments, removal of the precipate after performing acid precipitation on the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria) is facilitated by the use of centrifugation.

In some embodiments, ethylenediaminetetraacetic acid (EDTA) is added to the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria) before it is subjected to acid precipitation. For example, EDTA can be added to the liquid including the recombinant ranibizumab or ranibizumab variant (e.g., a liquid culture medium including recombinant ranibizumab or ranibizumab variant, and optionally, substantially free of recombinant bacteria) to a concentration of about 1 mM to about 10 mM (e.g., about 1 mM to about 2 mM, about 1 mM to about 4 mM, about 1 mM to about 6 mM, about 1 mM to about 8 mM, about 8 mM to about 10 mM, about 6 mM to about 10 mM, about 4 mM to about 10 mM, about 2 mM to about 10 mM, about 2 mM to about 4 mM, about 2 mM to about 6 mM, about 2 mM to about 8 mM, about 6 mM to about 8 mM, about 4 mM to about 8 mM, or about 4 mM to about 6 mM). In some embodiments, EDTA is added to the recombinant protein production medium to a concentration of about 2 mM, 2.5 mM, 3 mM, 3.5 mM, 4 mM, 4.5 mM, 5 mM, 5.5 mM, 6 mM, 6.5 mM, 7 mM, 7.5 mM, or about 8 mM.

In some embodiments, NaCl is added to the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria) before it is subjected to acid precipitation. For example, NaCl can be added to the liquid including the recombinant ranibizumab (e.g., a liquid culture medium including recombinant ranibizumab or ranibizumab variant, and optionally, substantially free of recombinant bacteria) to a concentration of about 0.2 M to about 0.6 M (e.g., about 0.2 M to about 0.6 M, about 0.25 M to about 0.6 M, about 0.3 M to about 0.6 M, about 0.35 M to about 0.6 M, about 0.4 M to about 0.6 M, about 0.45 M to about 0.6 M, about 0.5 M to about 0.6 M, about 0.55 M to about 0.6 M, about 0.5 M to about 0.6 M, about 0.45 M to about 0.6 M, about 0.4 M to about 0.6 M, about 0.35 M to about 0.6 M, about 0.3 M to about 0.6 M, about 0.25 M to about 0.6 M, about 0.25 M to about 0.45 M, about 0.3 M to about 0.5 M, or about 0.35 M to about 0.55 M. In some embodiments, NaCl is added to the recombinant protein production medium to a concentration of about 0.2 M, about 0.25 M, about 0.3 M, about 0.35 M, about 0.4 M, about 0.45 M, about 0.5 M, about 0.55 M, or about 0.6 M.

In some embodiments, the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria) recovered after the acid precipitation step is adjusted to a pH of about 4.5 to about 6, e.g., after a period of time. For example, the pH is adjusted to a pH of about 4.5 to about 4.7, about 4.5 to about 4.9, about 4.5 to about 5.1, about 4.5 to about 5.3, about 4.5 to about 5.5, about 4.5 to about 5.7, about 4.8 to about 6, about 4.6 to about 6, or about 4.8 to about 5.2. In some embodiments, the liquid including the recombinant protein (e.g., ranibizumab or a ranibizumab variant) (e.g., a liquid culture medium including recombinant protein (e.g., ranibizumab or a ranibizumab variant), and optionally, substantially free of recombinant bacteria) recovered after the acid precipitation step is adjusted to a pH of about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6.

Filtering and Clarifying

In some embodiments, the method of purifying recombinant protein (e.g., ranibizumab or a ranibizumab variant) can include, at least in part, a step of filtering or clarifying a liquid including recombinant protein (e.g., ranibizumab or a ranibizumab variant) (e.g., a liquid culture medium including recombinant protein (e.g., ranibizumab or a ranibizumab variant), and optionally, substantially free of recombinant bacteria, a diluted liquid culture medium, or a processed liquid culture medium, such as a liquid including recombinant protein (e.g., ranibizumab or a ranibizumab variant) generated after acid precipitation). The filtration can be performed, e.g., using a filter, or a chromatography column or chromatographic membrane that contains a molecule sieve resin.

Capturing

In some embodiments, recombinant protein (e.g., ranibizumab or a ranibizumab variant) can be purified using, at least in part, a step of capturing recombinant protein (e.g., ranibizumab or a ranibizumab variant) from a liquid (e.g., a liquid culture medium substantially free of recombinant bacteria, a diluted liquid culture medium, or a processed liquid culture medium such as an acid-precipitated and/or clarified liquid culture medium). Capturing ranibizumab or a ranibizumab variant can include the use of, e.g., a chromatography column or a chromatography resin, e.g., that utilizes a capture mechanism. Non-limiting examples of capturing mechanisms include an antibody- or antibody fragment-binding capture mechanism, an aptamer-binding capture mechanism, and a protein L-binding capture mechanism. In some embodiments, the capturing is performed using an antibody- or antibody fragment-binding capture mechanism. In some embodiments, the antibody- or antibody-fragment binding capturing mechanism can be a protein L- or a protein G-binding capture mechanism or an antigen-binding capturing mechanism. In some embodiments, the antibody- or antibody-fragment binding capture mechanism is a kappa light chain-binding capture mechanism (e.g., CAPTO® Protein L chromatography resin). In order to capture recombinant protein (e.g., ranibizumab or a ranibizumab variant) using the chromatography column or chromatographic membrane, one typically performs the sequential chromatographic steps of loading, washing, eluting, and regenerating the chromatography column or chromatography membrane. As one of skill in the art can appreciate, the flow rates and buffers to be used loading, washing, eluting, and regenerating steps are chosen based on the chemical properties of the recombinant protein (e.g., ranibizumab or a ranibizumab variant) and the chromatographic resin used to perform the capturing.

Following the loading of recombinant protein (e.g., ranibizumab or a ranibizumab variant) onto the chromatographic column or chromatographic membrane that is used to perform the unit operation of capturing recombinant protein (e.g., ranibizumab or a ranibizumab variant), the chromatographic column or chromatographic membrane can be washed with at least one washing buffer. As can be appreciated in the art, the washing buffer is meant to elute all proteins that are not the recombinant protein (e.g., ranibizumab or a ranibizumab variant) from the chromatography column or chromatographic membrane, while not disturbing the interaction of the recombinant protein (e.g., ranibizumab or a ranibizumab variant) with the resin or otherwise eluting the recombinant protein (e.g., ranibizumab or a ranibizumab variant).

A non-limiting example of a wash buffer that can be used in the capturing step includes about 5 mM to about 100 mM (e.g., about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 5 mM to about 10 mM, about 10 mM to about 50 mM, about 10 mM to about 40 mM, about 10 mM to about 30 mM, about 10 mM to about 20 mM, about 15 mM to about 50 mM, about 15 mM to about 40 mM, about 15 mM to about 30 mM, about 15 mM to about 20 mM, about 20 mM to about 50 mM, about 20 mM to about 40 mM, about 20 mM to about 30 mM, about 25 mM to about 50 mM, about 25 mM to about 40 mM, about 25 mM to about 30 mM, about 30 mM to about 50 mM, about 30 mM to about 40 mM, about 35 mM to about 50 mM, about 35 mM to about 40 mM, about 40 mM to about 50 mM, or about 45 mM to about 50 mM) buffer (e.g., acetate), about 0.1 mM to about 2 mM (e.g., about 0.1 mM to about 1.8 mM, about 0.1 mM to about 1.6 mM, about 0.1 mM to about 1.4 mM, about 0.1 mM to about 1.2 mM, about 0.1 mM to about 1.0 mM, about 0.1 mM to about 0.8 mM, about 0.1 mM to about 0.6 mM, about 0.1 mM to about 0.4 mM, about 0.1 mM to about 0.2 mM, about 0.2 mM to about 2.0 mM, about 0.2 mM to about 1.8 mM, about 0.2 mM to about 1.6 mM, about 0.2 mM to about 1.4 mM, about 0.2 mM to about 1.0 mM, about 0.2 mM to about 0.8 mM, about 0.2 mM to about 0.6 mM, about 0.2 mM to about 0.4 mM, about 0.4 mM to about 2.0 mM, about 0.4 mM to about 1.8 mM, about 0.4 mM to about 1.6 mM, about 0.4 mM to about 1.4 mM, about 0.4 mM to about 1.2 mM, about 0.4 mM to about 1.0 mM, about 0.4 mM to about 0.8 mM, about 0.4 mM to about 0.6 mM, about 0.6 mM to about 2.0 mM, about 0.6 mM to about 1.8 mM, about 0.6 mM to about 1.6 mM, about 0.6 mM to about 1.4 mM, about 0.6 mM to about 1.2 mM, about 0.6 mM to about 1.0 mM, about 0.6 mM to about 0.8 mM, about 0.8 mM to about 2.0 mM, about 0.8 mM to about 1.8 mM, about 0.8 mM to about 1.6 mM, about 0.8 mM to about 1.4 mM, about 0.8 mM to about 1.2 mM, about 0.8 mM to about 1.0 mM, about 1.0 mM to about 2.0 mM, about 1.0 mM to about 1.8 mM, about 1.0 mM to about 1.6 mM, about 1.0 mM to about 1.4 mM, about 1.0 mM to about 1.2 mM, about 1.2 mM to about 2.0 mM, about 1.2 mM to about 1.8 mM, about 1.2 mM to about 1.6 mM, about 1.2 mM to about 1.4 mM, about 1.4 mM to about 2.0 mM, about 1.4 mM to about 1.8 mM, about 1.4 mM to about 1.6 mM, about 1.6 mM to about 2.0 mM, about 1.6 mM to about 1.8 mM, or about 1.8 mM to about 2.0 mM) chelator (e.g., EDTA), and about 200 mM to about 500 mM (e.g., about 200 mM to about 480 mM, about 200 mM to about 460 mM, about 200 mM to about 440 mM, about 200 mM to about 420 mM, about 200 mM to about 400 mM, about 200 mM to about 380 mM, about 200 mM to about 360 mM, about 200 mM to about 340 mM, about 200 mM to about 320 mM, about 200 mM to about 300 mM, about 200 mM to about 280 mM, about 200 mM to about 260 mM, about 200 mM to about 240 mM, about 200 mM to about 200 mM, about 220 mM to about 500 mM, about 220 mM to about 480 mM, about 220 mM to about 460 mM, about 220 mM to about 440 mM, about 220 mM to about 420 mM, about 220 mM to about 400 mM, about 220 mM to about 380 mM, about 220 mM to about 360 mM, about 220 mM to about 340 mM, about 220 mM to about 320 mM, about 220 mM to about 300 mM, about 220 mM to about 280 mM, about 220 mM to about 260 mM, about 220 mM to about 240 mM, about 240 mM to about 500 mM, about 240 mM to about 480 mM, about 240 mM to about 460 mM, about 240 mM to about 440 mM, about 240 mM to about 420 mM, about 240 mM to about 400 mM, about 240 mM to about 380 mM, about 240 mM to about 360 mM, about 240 mM to about 340 mM, about 240 mM to about 320 mM, about 240 mM to about 300 mM, about 240 mM to about 280 mM, about 240 mM to about 260 mM, about 260 mM to about 500 mM, about 260 mM to about 480 mM, about 260 mM to about 460 mM, about 260 mM to about 440 mM, about 260 mM to about 420 mM, about 260 mM to about 400 mM, about 260 mM to about 380 mM, about 260 mM to about 360 mM, about 260 mM to about 340 mM, about 260 mM to about 320 mM, about 260 mM to about 300 mM, about 260 mM to about 280 mM, about 280 mM to about 500 mM, about 280 mM to about 480 mM, about 280 mM to about 460 mM, about 280 mM to about 440 mM, about 280 mM to about 420 mM, about 280 mM to about 400 mM, about 280 mM to about 380 mM, about 280 mM to about 360 mM, about 280 mM to about 340 mM, about 280 mM to about 320 mM, about 280 mM to about 300 mM, about 300 mM to about 500 mM, about 300 mM to about 480 mM, about 300 mM to about 460 mM, about 300 mM to about 440 mM, about 300 mM to about 420 mM, about 300 mM to about 400 mM, about 300 mM to about 380 mM, about 300 mM to about 360 mM, about 300 mM to about 340 mM, about 300 mM to about 320 mM, about 320 mM to about 500 mM, about 320 mM to about 480 mM, about 320 mM to about 460 mM, about 320 mM to about 440 mM, about 320 mM to about 420 mM, about 320 mM to about 400 mM, about 320 mM to about 380 mM, about 320 mM to about 360 mM, about 320 mM to about 340 mM, about 340 mM to about 500 mM, about 340 mM to about 480 mM, about 340 mM to about 460 mM, about 340 mM to about 440 mM, about 340 mM to about 420 mM, about 340 mM to about 400 mM, about 340 mM to about 380 mM, about 340 mM to about 360 mM, about 360 mM to about 500 mM, about 360 mM to about 480 mM, about 360 mM to about 460 mM, about 360 mM to about 440 mM, about 360 mM to about 420 mM, about 360 mM to about 400 mM, about 360 mM to about 380 mM, about 380 mM to about 500 mM, about 380 mM to about 480 mM, about 380 mM to about 460 mM, about 380 mM to about 440 mM, about 380 mM to about 420 mM, about 380 mM to about 400 mM, about 400 mM to about 500 mM, about 400 mM to about 480 mM, about 400 mM to about 460 mM, about 400 mM to about 440 mM, about 400 mM to about 420 mM, about 420 mM to about 500 mM, about 420 mM to about 480 mM, about 420 mM to about 460 mM, about 420 mM to about 440 mM, about 440 mM to about 500 mM, about 440 mM to about 480 mM, about 440 mM to about 460 mM, about 460 mM to about 500 mM, about 460 mM to about 480 mM, or about 480 mM to about 500 mM) salt (e.g., NaCl), and has a pH of about 4.0 to about 5.0 (e.g., about 4.0 to about 4.8, about 4.0 to about 4.6, about 4.0 to about 4.4, about 4.0 to about 4.2, about 4.2 to about 5.0, about 4.2 to about 4.8, about 4.2 to about 4.6, about 4.2 to about 4.4, about 4.4 to about 5.0, about 4.4 to about 4.8, about 4.4 to about 4.6, about 4.6 to about 5.0, about 4.6 to about 4.8, or about 4.8 to about 5.0).

Non-limiting examples of elution buffers used in these methods will depend on the resin used to perform the capturing. For example, an elution buffer can contain a different concentration of salt (e.g., increased salt concentration), a different pH (e.g., an increased or decreased pH), or a molecule that will compete with the recombinant protein (e.g., ranibizumab or a ranibizumab variant) for binding to the resin. Examples of such elution buffers are known in the art. In some embodiments, the recombinant protein (e.g., ranibizumab or a ranibizumab variant) is eluted with a low pH, low salt buffer (e.g, a buffer with a pH of about 2.5 to about 4).

A non-limiting example an elution buffer that can be used in the capturing step can include about 5 mM to about 100 mM (e.g., any of the subranges of this range described herein) buffer (e.g., acetate), about 0.1 mM to about 2 mM (e.g., any of the subranges of this range described herein) chelator (e.g., EDTA), 0 to about 10 mM (e.g., 0 to about 8 mM, 0 to about 6 mM, 0 to about 4 mM, 0 to about 2 mM, 0 to about 1 mM, about 0 to about 0.5 mM, about 0.5 mM to about 10 mM, about 0.5 mM to about 8 mM, about 0.5 mM to about 6 mM, about 0.5 mM to about 4 mM, about 0.5 mM to about 2 mM, about 0.5 mM to about 1 mM, about 1 mM to about 10 mM, about 1 mM to about 8 mM, about 1 mM to about 6 mM, about 1 mM to about 4 mM, about 1 mM to about 2 mM, about 2 mM to about 10 mM, about 2 mM to about 8 mM, about 2 mM to about 6 mM, about 2 mM to about 4 mM, about 4 mM to about 10 mM, about 4 mM to about 8 mM, about 4 mM to about 6 mM, about 6 mM to about 10 mM, about 6 mM to about 8 mM, or about 8 mM to about 10 mM) salt (e.g., NaCl).

In some embodiments, the yield for the capture step (% recombinant protein recovered as compared to the recombinant protein loaded) is from about 30% to about 100%. In some embodiments, the yield for the capture step is from about 40% to about 90%. In some embodiments, the yield for the capture step is from about 50% to about 70%. In some embodiments, the yield for the capture step is from about 50% to about 60%. In some embodiments, the yield for the capture step is about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Following the elution of the recombinant protein (e.g., ranibizumab or a ranibizumab variant) from the chromatographic column or chromatographic membrane used to perform the unit operation of purifying the recombinant protein (e.g., ranibizumab or a ranibizumab variant), the chromatography column or chromatographic membrane can be equilibrated using a regeneration buffer. In some embodiments, before the next step in the process, the eluate from the chromatographic column or chromatographic membrane is diluted about 0.1-fold to about 10-fold (e.g., about 0.1-fold to about 8-fold, about 0.1-fold to about 6-fold, about 0.1-fold to about 4-fold, about 0.1-fold to about 2-fold, about 0.1-fold to about 1-fold, about 1-fold to about 10-fold, about 1-fold to about 8-fold, about 1-fold to about 6-fold, about 1-fold to about 4-fold, about 1-fold to about 2-fold, about 2-fold to about 10-fold, about 2-fold to about 8-fold, about 2-fold to about 6-fold, about 2-fold to about 4-fold, about 4-fold to about 10-fold, about 4-fold to about 8-fold, about 4-fold to about 6-fold, about 6-fold to about 10-fold, about 6-fold to about 8-fold, or about 8-fold to about 10-fold).

Purifying

In some embodiments, the methods of purifying the recombinant protein (e.g., ranibizumab or a ranibizumab variant) can include one or more steps of purifying the recombinant protein (e.g., ranibizumab or a ranibizumab variant) using one or more chromatography columns or chromatographic membranes that contains a resin, e.g., that binds the recombinant protein (e.g., ranibizumab or a ranibizumab variant). For example, the chromatography column(s) or chromatography membrane(s) can be used to perform ionic exchange chromatography, such as cation exchange chromatography.

In some embodiments, a purifying step separates an antibody fragment (e.g., a Fab fragment, a F(ab′)2 fragment, or an a scFv fragment) from other light-chain containing proteins. In one embodiment, a purifying step separates a Fab from other light-chain containing proteins. In another embodiment, a purifying step separates ranibizumab or a ranibizumab variant from other light-chain containing proteins.

In some embodiments, an antibody fragment is separated from light-chain containing proteins in a purifying step by binding the antibody fragment to a resin under conditions where other light chain containing proteins wash through. In some embodiments, an antibody fragment is separated from light-chain containing proteins in a purifying step by binding the antibody fragment to a resin under conditions where other light chain containing proteins are also bound the resin then selectively eluted while the antibody fragment remains bound to the resin. In some embodiments, an antibody fragment is separated from light-chain containing proteins in a purifying step by binding the antibody fragment to a resin under conditions where other light chain containing proteins are also bound the resin then selectively eluting the antibody fragment while the other light chain containing proteins remain bound to the resin.

In some embodiments, the yield for the purifying step (% recombinant protein recovered as compared to the recombinant protein loaded) is from about 10% to about 100%. In some embodiments, the yield for the purifying step is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the methods of purifying the recombinant protein (e.g., ranibizumab or a ranibizumab variant) can include a first cation exchange chromatography column and a second cation exchange chromatography column. As can be appreciated by one skilled in the art, the step of purifying recombinant protein (e.g., ranibizumab or a ranibizumab variant) can, e.g., include the steps of loading, washing, eluting, and equilibrating the first and/or second cation exchange chromatography column or chromatographic membrane used to perform the unit of operation of purifying recombinant protein (e.g., ranibizumab or a ranibizumab variant). Typically, the eluate coming out of a chromatography column or chromatographic membrane used to perform the unit operation of purifying contains the recombinant protein (e.g., ranibizumab or a ranibizumab variant).

Following the loading of the recombinant protein (e.g., ranibizumab or a ranibizumab variant) onto the chromatographic column or chromatographic membrane that is used to perform the unit operation of purifying the recombinant protein (e.g., ranibizumab or a ranibizumab variant), the chromatographic column or chromatographic membrane is washed with at least one washing buffer. As can be appreciated in the art, the washing buffer is meant to elute all proteins that are not the recombinant protein (e.g., ranibizumab or a ranibizumab variant) from the chromatography column or chromatographic membrane, while not disturbing the interaction of the recombinant protein (e.g., ranibizumab or a ranibizumab variant) with the resin or otherwise eluting the recombinant protein (e.g., ranibizumab or a ranibizumab variant).

In some embodiments, the step of purifying the recombinant protein (e.g., ranibizumab or a ranibizumab variant) in the eluate of the affinity chromatography column using a first cation exchange chromatography column is performed using buffered solutions having a pH of about 6 to about 6.8 (e.g., about 6 to about 6.1, about 6 to about 6.2, about 6 to about 6.3, about 6 to about 6.4, about 6 to about 6.5, about 6 to about 6.6, about 6 to about 6.7, about 6.7 to about 6.8, about 6.6 to about 6.8, about 6.5 to about 6.8, about 6.4 to about 6.8, about 6.3 to about 6.8, about 6.2 to about 6.8, about 6.1 to about 6.8, about 6.2 to about 6.4, about 6.3 to about 6.5, about 6.4 to about 6.6, about 6.5 to about 6.7, or about 6.6 to about 6.7). In some embodiments, the step of purifying the recombinant protein (e.g., ranibizumab or a ranibizumab variant) in the eluate of the affinity chromatography column using the first cation exchange chromatography column is performed using buffered solutions having a pH of about 6, about 6.1, about 6.2, about 6.3, about 6.4, about 6.5, about 6.6, about 6.7, or about 6.8.

In some embodiments, the wash buffer used in the first cation exchange chromatography column can include: about 1 mM to about 100 mM (e.g., about 1 mM to about 90 mM, about 1 mM to about 80 mM, about 1 mM to about 70 mM, about 1 mM to about 60 mM, about 1 mM to about 50 mM, about 1 mM to about 40 mM, about 1 mM to about 30 mM, about 1 mM to about 20 mM, about 1 mM to about 10 mM, about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 5 mM to about 10 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 100 mM, about 50 mM to about 90 mM, about 50 mM to about 80 mM, about 50 mM to about 70 mM, about 50 mM to about 60 mM, about 60 mM to about 100 mM, about 60 mM to about 90 mM, about 60 mM to about 80 mM, about 60 mM to about 70 mM, about 70 mM to about 100 mM, about 70 mM to about 90 mM, about 70 mM to about 80 mM, about 80 mM to about 100 mM, about 80 mM to about 90 mM, or about 90 mM to about 100 mM) buffer (e.g., a Good's buffer, e.g., MES); and 0.1 mM to about 2 mM (e.g., any of the subranges of this range described herein) chelator (e.g., EDTA); and having a pH of about 6.0 to about 6.8 (e.g., any of the subranges of this range described herein).

In some embodiments, the yield for the first cation exchange chromatography column (% recombinant protein recovered as compared to the recombinant protein loaded) is from about 10% to about 90%. In some embodiments, the yield for the first cation exchange chromatography column is from about 15% to about 40%. In some embodiments, the yield for the first cation exchange chromatography column is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%.

In some embodiments, the elution buffer used in the first cation exchange chromatography column can include: about 1 mM to about 100 mM (e.g., any of the subranges of this range described herein) buffer (e.g., a Good's buffer, e.g., MES); 0.1 mM to about 2.0 mM (e.g., any of the subranges of this range described herein) chelator (e.g., EDTA); and a pH of about 6.0 to about 6.8 (e.g., any of the subranges of this range described herein).

Some embodiments further include adjusting ionic concentration of the eluate of the first cation exchange chromatography column after purifying the recombinant protein (e.g., ranibizumab or a ranibizumab variant) in the eluate of the affinity chromatography column using a first cation exchange chromatography column and before purifying the recombinant protein (e.g., ranibizumab or a ranibizumab variant) in the eluate from the first cation exchange chromatography column using a second cation chromatography column. Some embodiments further include diluting the eluate of the first cation exchange chromatography column after purifying the recombinant protein (e.g., ranibizumab or a ranibizumab variant) in the eluate of the affinity chromatography column using a first cation exchange chromatography column and before purifying the recombinant protein (e.g., ranibizumab or a ranibizumab variant) in in the eluate from the first cation exchange chromatography column using a second cation chromatography column. For example, the eluate of the first cation exchange chromatography column can be diluted with an equal volume of water for injection.

For example, the eluate including recombinant protein (e.g., ranibizumab or a ranibizumab variant) is diluted, e.g., about 0.1-fold to about 10-fold (e.g., about 0.1-fold to about 8-fold, about 0.1-fold to about 6-fold, about 0.1-fold to about 4-fold, about 0.1-fold to about 2-fold, about 0.1-fold to about 1-fold, about 0.1-fold to about 0.5-fold, about 0.2-fold to about 10-fold, about 0.2-fold to about 8-fold, about 0.2-fold to about 6-fold, about 0.2-fold to about 4-fold, about 0.2-fold to about 2-fold, about 0.2-fold to about 1-fold, about 0.2-fold to about 0.5-fold, about 0.5-fold to about 10-fold, about 0.5-fold to about 8-fold, about 0.5-fold to about 6-fold, about 0.5-fold to about 4-fold, about 0.5-fold to about 2-fold, about 0.5-fold to about 1-fold, about 1-fold to about 10-fold, about 1-fold to about 8-fold, about 1-fold to about 6-fold, about 1-fold to about 4-fold, about 1-fold to about 2-fold, about 2-fold to about 10-fold, about 2-fold to about 8-fold, about 2-fold to about 6-fold, about 2-fold to about 4-fold, about 4-fold to about 10-fold, about 4-fold to about 8-fold, about 4-fold to about 6-fold, about 6-fold to about 10-fold, about 6-fold to about 8-fold, or about 8-fold to about 10-fold) before it is loaded onto the second cation exchange chromatography resin.

In some embodiments, the step of purifying the recombinant protein (e.g., ranibizumab or a ranibizumab variant) in the eluate from the first cation exchange chromatography column using the second cation exchange chromatography column is performed using buffered solutions having a pH of about 4.5 to about 5.4, e.g., about 4.5 to about 4.6, about 4.5 to about 4.7, about 4.5 to about 4.8, about 4.5 to about 4.9, about 4.5 to about 5, about 4.5 to about 5.1, about 4.5 to about 5.2, about 4.5 to about 5.3, about 5.3 to about 5.4, about 5.3 to about 5.4, about 5.2 to about 5.1, about 5 to about 5.4, about 4.9 to about 5.4, about 4.8 to about 5.4, about 4.7 to about 5.4, about 4.6 to about 5.4, about 4.6 to about 4.8, about 4.7 to about 4.9, about 4.8 to about 5, about 4.8 to about 5.2, about 4.9 to about 5.1, about 5 to about 5.2, about 5.1 to about 5.3, about 5.2 to about 5.4, or about 5.3 to about 5.5. In some embodiments, the step of purifying the recombinant protein (e.g., ranibizumab or a ranibizumab variant) in the eluate from the first cation exchange chromatography column using the second cation exchange chromatography column is performed using buffered solutions having a pH of about 4.5, about 4.6, about 4.7, about 4.8, about 4.9, about 5.0, about 5.4, about 5.2, about 5.3, or about 5.4.

In some embodiments, the wash buffer used in the second cation exchange chromatography column can include: about 1 mM to about 100 mM (e.g., any of the subranges of this range described herein) buffer (e.g., acetate); about 0.1 mM to about 2.0 mM (e.g., any of the subranges of this range described herein) chelator (e.g., EDTA); and about 0 to about 40 mM (e.g., 0 to about 35 mM, 0 to about 30 mM, 0 to about 25 mM, 0 to about 20 mM, 0 to about 15 mM, 0 to about 10 mM, 0 to about 5 mM, 0 to 2 mM, 0 to about 1 mM, 0 to about 0.5 mM, 0 to about 0.2 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 5 mM, about 0.1 mM to 2 mM, about 0.1 mM to about 1 mM, about 0.1 mM to about 0.5 mM, about 0.1 mM to about 0.2 mM, about 0.2 mM to about 40 mM, about 0.2 mM to about 35 mM, about 0.2 mM to about 30 mM, about 0.2 mM to about 25 mM, about 0.2 mM to about 20 mM, about 0.2 mM to about 15 mM, about 0.2 mM to about 10 mM, about 0.2 mM to about 5 mM, about 0.2 mM to 2 mM, about 0.2 mM to about 1 mM, about 0.2 mM to about 0.5 mM, about 0.5 mM to about 40 mM, about 0.5 mM to about 35 mM, about 0.5 mM to about 30 mM, about 0.5 mM to about 25 mM, about 0.5 mM to about 20 mM, about 0.5 mM to about 15 mM, about 0.5 mM to about 10 mM, about 0.5 mM to about 5 mM, about 0.5 mM to 2 mM, about 0.5 mM to about 1 mM, about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 5 mM, about 1 mM to 2 mM, about 2 mM to about 40 mM, about 2 mM to about 35 mM, about 2 mM to about 30 mM, about 2 mM to about 25 mM, about 2 mM to about 20 mM, about 2 mM to about 15 mM, about 2 mM to about 10 mM, about 2 mM to about 5 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 40 mM, about 15 mM to about 35 mM, about 15 mM to about 30 mM, about 15 mM to about 25 mM, about 15 mM to about 20 mM, about 20 mM to about 40 mM, about 20 mM to about 35 mM, about 20 mM to about 30 mM, about 20 mM to about 25 mM, about 25 mM to about 40 mM, about 25 mM to about 35 mM, about 25 mM to about 30 mM, about 30 mM to about 40 mM, about 30 mM to about 35 mM, or about 35 mM to about 40 mM) salt (e.g., NaCl); and having a pH of about 4.5 to about 5.4 (e.g., any of the subranges of this range described herein).

In some embodiments, the elution buffer used in the second cation exchange chromatography column can include: about 1 mM to about 100 mM (e.g., any of the subranges of this range described herein) buffer (e.g., acetate); about 0.1 mM to about 2.0 mM (e.g., any of the subranges of this range described herein) chelator (e.g., EDTA); and about 40 mM to about 140 mM (e.g., about 40 mM to about 120 mM, about 40 mM to about 100 mM, about 40 mM to about 80 mM, about 40 mM to about 60 mM, about 60 mM to about 140 mM to about 60 mM to about 120 mM, about 60 mM to about 100 mM, about 60 mM to about 80 mM, about 80 mM to about 140 mM, about 80 mM to about 120 mM, about 80 mM to about 100 mM, about 100 mM to about 140 mM, about 100 mM to about 120 mM, or about 120 mM to about 140 mM) NaCl; and having a pH of about 4.5 to about 5.4 (e.g., any of the subranges of this range described herein).

In some embodiments, the yield for the second cation exchange chromatography column (% recombinant protein recovered as compared to the recombinant protein loaded) is from about 50% to about 100%. In some embodiments, the yield for the second cation exchange chromatography column is from about 65% to about 99%. In some embodiments, the yield for the second cation exchange chromatography column is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the first cation exchange chromatography column comprises a CAPTO™ SP ImpRes chromatography resin. In some embodiments, the second cation exchange chromatography column comprises a CAPTO™ SP ImpRes chromatography resin.

Following the elution of the recombinant protein (e.g., ranibizumab or a ranibizumab variant) from the chromatographic column or chromatographic membrane used to perform the unit operation of purifying the recombinant protein (e.g., ranibizumab or a ranibizumab variant), the chromatography column or chromatographic membrane can be equilibrated using a regeneration buffer.

Some embodiments further include filtering the eluate from the second cation exchange chromatography column after purifying the recombinant protein (e.g., ranibizumab or a ranibizumab variant) in the eluate from the first cation exchange chromatography column using a second cation chromatography column.

Some embodiments further include adjusting ionic concentration of the eluate of the second cation exchange chromatography column after purifying the recombinant protein (e.g., ranibizumab or a ranibizumab variant) in the eluate from the first cation exchange chromatography column using a second cation chromatography column. Some embodiments further include diluting the eluate of the second cation exchange chromatography column after purifying the recombinant protein (e.g., ranibizumab or a ranibizumab variant) in the eluate from the first cation exchange chromatography column using a second cation chromatography column.

In some embodiments, the eluate from the second cation exchange chromatography column is conditioned to final concentration of about 0.5 M to about 3.0 M (e.g., 0.5 M to about 2.5 M, about 0.5 M to about 2.0 M, about 0.5 M to about 1.5 M, about 0.5 M to about 1.0 M, about 1.0 M to about 2.5 M, about 1.0 M to about 2.0 M, about 1.0 M to about 1.5 M, about 1.5 M to about 2.5 M, about 1.5 M to about 2.0 M, or about 2.0 M to about 2.5 M) salt (e.g., NaCl), before it is loaded onto a chromatography resin (e.g., a hydrophobic chromatography resin) for polishing the recombinant protein (e.g., ranibizumab or a ranibizumab variant).

Polishing

In some embodiments, the methods of purifying the recombinant protein (e.g., ranibizumab or a ranibizumab variant) can further include a step polishing the recombinant the recombinant protein (e.g., ranibizumab or a ranibizumab variant). The recombinant protein (e.g., ranibizumab or a ranibizumab variant) can be polished using, e.g., a chromatography column or chromatographic membrane that contains a hydrophobic interaction chromatography resin. Hydrophobic interaction resins can be used to remove product-related charge variants, host-cell impurities, endotoxin, or a combination thereof. Non-limiting examples of hydrophobic interaction chromatography resins are known in the art. In some embodiments, the hydrophobic interaction chromatography column comprises a phenyl sepharose chromatography resin.

As can be appreciated in the art, polishing recombinant protein (e.g., ranibizumab or a ranibizumab variant) using the chromatography column or chromatography membrane can include, e.g., the steps of loading, washing, eluting, and equilibrating the at least one chromatography column or chromatographic membrane used to perform the unit of operation of polishing the recombinant protein (e.g., ranibizumab or a ranibizumab variant). Typically, the elution buffer coming out of a chromatography column or chromatographic membrane used to perform the unit operation of polishing contains recombinant protein (e.g., ranibizumab or a ranibizumab variant). In some embodiments, the recombinant protein (e.g., ranibizumab or a ranibizumab variant) is eluted using a gradient of decreasing ionic strength.

For example, the wash buffer used in the hydrophobic interaction chromatography column can include: about 1 mM to about 100 mM (e.g., any of the subranges of this range described herein) buffer (e.g., histidine); and about 0.5 M to about 3.0 M (e.g., any of the subranges of this range described herein) salt (e.g., NaCl); and having a pH of about 5.0 to about 6.0 (e.g., about 5.0 to about 5.9, about 5.0 to about 5.8, about 5.0 to about 5.7, about 5.0 to about 5.6, about 5.0 to about 5.5, about 5.0 to about 5.4, about 5.0 to about 5.3, about 5.0 to about 5.2, about 5.0 to about 5.1, about 5.1 to about 6.0, about 5.1 to about 5.9, about 5.1 to about 5.8, about 5.1 to about 5.7, about 5.1 to about 5.6, about 5.1 to about 5.5, about 5.1 to about 5.4, about 5.1 to about 5.3, about 5.1 to about 5.2, about 5.2 to about 6.0, about 5.2 to about 5.9, about 5.2 to about 5.8, about 5.2 to about 5.7, about 5.2 to about 5.6, about 5.2 to about 5.6, about 5.2 to about 5.5, about 5.2 to about 5.4, about 5.2 to about 5.3, about 5.3 to about 6.0, about 5.3 to about 5.9, about 5.3 to about 5.8, about 5.3 to about 5.7, about 5.3 to about 5.6, about 5.3 to about 5.5, about 5.3 to about 5.4, about 5.4 to about 6.0, about 5.4 to about 5.9, about 5.4 to about 5.8, about 5.4 to about 5.7, about 5.4 to about 5.6, about 5.4 to about 5.5, about 5.5 to about 6.0, about 5.5 to about 5.9, about 5.5 to about 5.8, about 5.5 to about 5.7, about 5.5 to about 5.6, about 5.6 to about 6.0, about 5.6 to about 5.9, about 5.6 to about 5.8, about 5.6 to about 5.7, about 5.7 to about 6.0, about 5.7 to about 5.9, about 5.7 to about 5.8, about 5.8 to about 6.0, about 5.8 to about 5.9, or about 5.9 to about 6.0).

For example, the elution buffer used in the hydrophobic interaction chromatography column can include: about 1 mM to about 100 mM (e.g., any of the subranges of this range described herein) buffer (e.g., histidine); about 0 to about 2.5 M (e.g., 0 to about 2.0 M, 0 to about 1.5 M, 0 to about 1.0 M, 0 to about 0.5 M, 0 to about 0.2 M, 0 to about 0.1 M, about 0.1 M to about 2.5 M, about 0.1 M to about 2.0 M, about 0.1 M to about 1.5 M, about 0.1 M to about 1.0 M, about 0.1 M to about 0.5 M, about 0.1 M to about 0.2 M, about 0.2 M to about 2.5 M, about 0.2 M to about 2.0 M, about 0.2 M to about 1.5 M, about 0.2 M to about 1.0 M, about 0.2 M to about 0.5 M, about 0.5 M to about 2.5 M, about 0.5 M to about 2.0 M, about 0.5 M to about 1.5 M, about 0.5 M to about 1.0 M, about 1.0 M to about 2.5 M, about 1.0 M to about 2.0 M, about 1.0 M to about 1.5 M, about 1.5 M to about 2.5 M, about 1.5 M to about 2.0 M, or about 2.0 M to about 2.5 M) salt (e.g., NaCl).

In some embodiments, the yield for the polishing step (% recombinant protein recovered as compared to the recombinant protein loaded) is from about 50% to about 100%. In some embodiments, the yield for the polishing step is from about 75% to about 99%. In some embodiments, the yield for the polishing step is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the yield of recombinant protein recovered from a purifying step or polishing step as compared to the recombinant protein loaded at the capture step is from about 1% to about 90%. In some embodiments, the yield for preparing drug substance is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%.

Ultrafiltration and Diafiltration

In some embodiments, the methods of purifying and polishing the recombinant protein (e.g., ranibizumab or a ranibizumab variant) can further include a step of filtering, diafiltering, concentrating, or a combination thereof. In some embodiments, the recombinant protein (e.g., ranibizumab or a ranibizumab variant) is buffer exchanged (e.g., diafiltered) into a formulation buffer. Some embodiments further include conditioning the eluate from the hydrophobic interaction chromatography column by diluting the eluate with an equal volume of water for injection prior to diafiltration. In some embodiments, the eluate from the hydrophobic interaction chromatography column is diluted with water for injection to about 10 mg/mL to about 14 mg/mL ranibizumab or ranibizumab variant (e.g., about 10 mg/mL, about 11 mg/mL, about 12 mg/mL, about 13 mg/mL, or about 14 mg/mL ranibizumab or ranibizumab variant). In some embodiments, the eluate is diafiltered against a formulation buffer lacking polysorbate. In some embodiments, the eluate is tangential flow filtered.

In some embodiments, the yield for this step (% recombinant protein recovered as compared to the recombinant protein loaded) is from about 70% to about 100%. In some embodiments, the yield for this step is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the yield of recombinant protein recovered from a step comprising filtering, diafiltering, concentrating, or a combination thereof as compared to the recombinant protein loaded at the capture step is from about 1% to about 90%. In some embodiments, the yield for preparing drug substance is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%.

### Pharmaceutical Compositions

Also provided herein are pharmaceutical compositions that include recombinant protein (e.g., ranibizumab or a ranibizumab variant) produced using any of the methods provided herein. The pharmaceutical compositions may be formulated in any manner described in the art. Pharmaceutical compositions are formulated to be compatible with their intended route of administration (e.g., intraarterial, intradermal, intramuscular, intravenous, intraperitoneal, or subcutaneous), and in dosage unit form (i.e., physically discrete units containing a predetermined quantity of recombinant protein (e.g., ranibizumab or a ranibizumab variant) for ease of administration and uniformity of dosage). The pharmaceutical compositions can include a sterile diluent (e.g., sterile water, saline, or water for injection), a fixed oil, polyethylene glycol, glycerin, propylene glycol, or other synthetic solvents, antibacterial or antifungal agents (e.g., benzyl alcohol, methyl parabens, chlorobutanol, phenol, ascorbic acid, or thimerosal), antioxidants (e.g., ascorbic acid or sodium bisulfite), chelating agents (e.g., EDTA), buffers (e.g., acetate buffer, citrate buffer, or phosphate buffer), isotonic agents (e.g., sugars, polyalcohols (e.g, mannitol or sorbitol), or salts (e.g., sodium chloride), or any combination thereof.

Pharmaceutical compositions that include recombinant protein (e.g., ranibizumab or a ranibizumab variant) as produced by any of the methods described herein can include about 5 mg/mL to about 15 mg/mL, about 5 mg/mL to about 14 mg/mL, about 5 mg/mL to about 13 mg/mL, about 5 mg/mL to about 12 mg/mL, about 5 mg/mL to about 11 mg/mL, about 5 mg/mL to about 10 mg/mL, about 5 mg/mL to about 9 mg/mL, about 5 mg/mL to about 8 mg/mL, about 5 mg/mL to about 7 mg/mL, about 5 mg/mL to about 6 mg/mL, about 6 mg/mL to about 15 mg/mL, about 6 mg/mL to about 14 mg/mL, about 6 mg/mL to about 13 mg/mL, about 6 mg/mL to about 12 mg/mL, about 6 mg/mL to about 11 mg/mL, about 6 mg/mL to about 10 mg/mL, about 6 mg/mL to about 9 mg/mL, about 6 mg/mL to about 8 mg/mL, about 6 mg/mL to about 7 mg/mL, about 8 mg/mL to about 12 mg/mL, about 9 mg/mL to about 11 mg/mL, about 8 mg/mL to about 10 mg/mL, about 10 mg/mL to about 12 mg/mL, about 9 mg/mL to about 10 mg/mL, or about 10 mg/mL to about 11 mg/mL of recombinant protein (e.g., ranibizumab or a ranibizumab variant) produced by any of the methods described herein. Some embodiments of any of the pharmaceutical compositions described herein can further include a tonicity agent (e.g., α,α-trehalose dihydrate), a buffer (e.g., a histidine buffer), a surfactant (e.g., polysorbate 20), and water for injection, and have a pH of about 5.0 to about 7.5, about 5.0 to about 7.0, about 5.0 to about 6.5, about 5.0 to about 6.0, about 5.0 to about 5.5, about 5.5 to about 7.5, about 5.5 to about 7.0, about 5.5 to about 6.5, about 5.5 to about 6.0, about 6.0 to about 7.5, about 6.0 to about 7.0, about 6.0 to about 6.5, about 6.5 to about 7.5, about 6.5 to about 7.0, or about 7.0 to about 7.5. In some embodiments, any of the pharmaceutical compositions provided herein can be disposed in a glass vial (e.g., a single-use type I glass vial) or a syringe.

In some embodiments, any of the pharmaceutical compositions described herein include less than 100 ppm, less than 95 ppm, less than 90 ppm, less than 80 ppm, less than 70 ppm, less than 60 ppm, less than 50 ppm, less than 45 ppm, less than 40 ppm, less than 35 ppm, less than 30 ppm, less than 25 ppm, less than 20 ppm, less than 15 ppm, less than 10 ppm, or less than 5 ppm host cell protein.

### Methods of Treatment

Also provided herein are methods of treating a subject (e.g., a human) in need thereof that include administering to the subject a therapeutically effective amount of any of the pharmaceutical compositions described herein that include recombinant protein (e.g., ranibizumab or a ranibizumab variant) as produced by any of the methods described herein. In some embodiments of these methods, the subject has been identified or diagnosed as having wet age-related macular degeneration, diabetic macular edema, or macular edema following retinal vein occlusion (e.g., branch retinal vein occlusion or central retinal vein occlusion).

Some embodiments include administration of one or more doses of any of the pharmaceutical compositions described herein to the subject (e.g., two or more doses, four or more doses, six or more doses, eight or more doses, or ten or more doses).

In some embodiments of any of the methods described herein, the pharmaceutical composition can be administered by subcutaneous administration, intramuscular administration, intravenous administration, intraocular administration, intraarterial administration, or intraperitoneal administration.

### Kits

Also provided herein are kits that include at least one dose of any of the pharmaceutical compositions described herein. In some embodiments, the kits can further include an item for use in administering a pharmaceutical composition (e.g., any of the pharmaceutical compositions described herein) to the mammal (e.g., a human).

In some examples, the kit can further include a sterile glass vial, where the pharmaceutical composition is disposed within the sterile glass vial. In some embodiments, the kit can further include a syringe and the pharmaceutical composition is disposed within the syringe.

Some examples of the kits include one or more doses (e.g., at least two, at least three, at least four, at least five, at least six, at least seven, at least eight, at least nine, at least ten, at least eleven, at least twelve, at least thirteen, at least fourteen, at least twenty, at least thirty, at least forty, or at least fifty doses) (e.g., intravenous, intraperitoneal, subcutaneous, intramuscular, or intraocular doses) of any of the pharmaceutical compositions described herein. In some examples, the kit further includes instructions for administering the pharmaceutical composition (or a dose of the pharmaceutical composition) to a mammal (e.g., a human in need thereof, e.g., any of the exemplary subjects described herein).

Also included herein are kits that include a sterile vial of a lyophilized recombinant protein (e.g., ranibizumab or a ranibizumab variant) (e.g., protein (e.g., ranibizumab or a ranibizumab variant)) produced by any of the methods described herein) cake or powder, instructions for reconstituting the cake or powder, and instructions for administration (e.g., intravenous, intramuscular, intraperitoneal, intraarterial, or intraocular administration) of the reconstituted solution to a mammal (e.g., a human in need thereof or any of the exemplary subjects described herein).

In some embodiments, the kits include a pharmaceutical composition including recombinant protein (e.g., ranibizumab or a ranibizumab variant) produced by any of the methods described herein, and a composition including at least one additional therapeutic agent. In some embodiments, the kit further contains instructions for administering the pharmaceutical composition including recombinant protein (e.g., ranibizumab or a ranibizumab variant) produced by any of the methods described herein and a composition including at least one additional therapeutic agent to a mammal (e.g., a human in need thereof or any of the other exemplary subjects described herein).

### EXAMPLES

The disclosure is further described in the following examples, which do not limit the scope of the disclosure described in the claims.

### Example 1. Purification of Ranibizumab

Ranibizumab was purified from a liquid culture medium including ranibizumab using the methods described below.

### Preparation of the Acid Precipitated Supernatant (APS)

An EDTA (0.4 M) stock solution was added to a final concentration of 5 mM to inhibit potential protease activity to a harvest centrate from a culture of recombinant E. coli BL21 cells expressing ranibizumab. A NaCl (5 M) solution was then added to the harvest centrate to a final concentration of 0.4 M followed by a hydrochloric acid solution (1 M), which was added with continuous mixing to lower the pH of the harvest centrate to 4.0. The acidified harvest centrate was incubated for about 2 hours at room temperature.

### Clarification, pH Adjustment, and Depth Filtration

The APS was centrifuged at 7844 RPM and 20° C. using an Alfa Laval MBPX810 separation system. Following centrifugation, the pH of the APS supernatant was adjusted to pH 5.0, and the APS supernatant comprising ranibizumab was further clarified using depth filtration.

### Affinity Chromatography

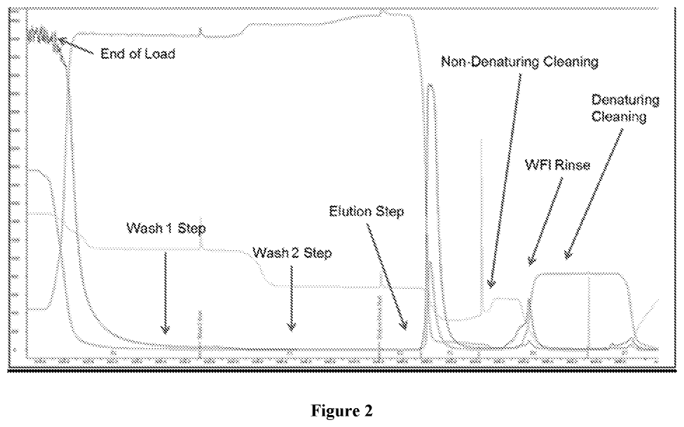

Affinity chromatography was used to bind ranibizumab via the kappa light chains. The clarified solution was loaded onto an equilibrated CAPTO™ L chromatography column. The column was then washed with 5 column volumes (CV) of 400 mM NaCl, 50 mM acetate, 1 mM EDTA, pH 5.0, and 5 CVs of 400 mM NaCl, 50 mM acetate, 1 mM EDTA, pH 4.0. Ranibizumab was eluted using a low pH, low salt step, i.e., 4 CVs of 50 mM acetate, 1 mM EDTA, pH 3.0. Table 1 provides a summary of the conditions for the CAPTO™ L chromatography step. FIG. 2 shows the real-time UV profile of the CAPTO™ L column over a limited time window.

TABLE 1
CAPTO ™ L Chromatography Conditions

| | | Step |
|---|---|---|
| | | Length |
| Process Step | Buffer | (CV) |
| Equilibration | 400 mM NaCl, 50 mM acetate, 1 mM EDTA, | 5 |
| | pH 5.0 | |
| Load | CAPS | NA |
| Wash 1 | 400 mM NaCl, 50 mM acetate, 1 mM EDTA, | 5 |
| | pH 5.0 | |
| Wash 2 | 400 mM NaCl, 50 mM acetate, 1 mM EDTA, | 5 |
| | pH 4.0 | |
| Elution | 50 mM acetate, 1 mM EDTA, pH 3.0 | 4 |
| Strip | 0.1M phosphoric acid | 3 |
| Rinse 1 | water for injection | 2 |
| Regeneration | 15 mM NaOH | 3 |
| Storage | 1% benzyl alcohol, 100 mM sodium acetate, | 3 |
| | pH 5.0 | |

### Ionic Exchange Chromatography, pH 6.5

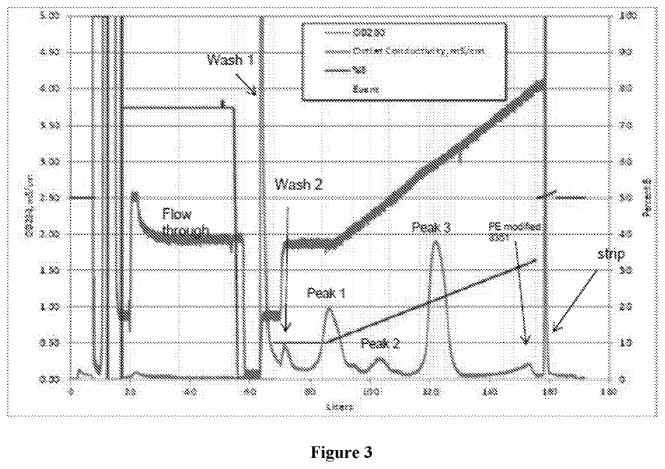

The eluate from the CAPTO™ L Column was diluted 4-fold with water for injection prior to loading onto a CAPTO™ SP ImpRes ion exchange column equilibrated at a pH of about 6.5. The ranibizumab was bound, a subset of impurities were removed by two isocratic washes, and the ranibizumab was eluted using a gradient of increasing ionic strength. Table 2 provides a summary of the conditions for the first ionic exchange chromatography step (at pH 6.5). FIG. 3 shows the real-time UV profile of the first ionic exchange column over a limited time window.

TABLE 2
CAPTO ™ SP ImpRes Chromatography Conditions, pH 6.5

| | | Step |
|---|---|---|
| | | Length |
| Process Step | Buffer | (CV) |
| Sanitization | 0.5M NaOH | 3 |
| Pre- | 20 mM MES, 100 mM NaCl, 0.1 mM EDTA, | 3 |
| Equilibration | pH 6.5 | |
| Equilibration | 20 mM MES, 0.1 mM EDTA, pH 6.5 | 3 |
| Load | 25% column 1 pool/75% water for injection | NA |
| Wash 1 | 20 mM MES, 0.1 mM EDTA, pH 6.5 | 3 |
| Wash 2 | 90%, 20 mM MES, 0.1 mM EDTA, pH 6.5 | 5 |
| | 10%, 100 mM NaCl, 20 mM MES, 0.1 mM | |
| | EDTA, pH 6.5 | |
| Elution | A buffer: 20 mM MES, 0.1 mM EDTA, pH 6.5 | 30 |
| | B buffer: 100 mM NaCl, 20 mM MES, 0.1 mM | |
| | EDTA, pH 6.5 | |
| | Linear gradient 10-40% B | |
| Strip | 20 mM MES, 100 mM NaCl, 0.1 mM EDTA, | 3 |
| | pH 6.5 | |
| Sanitization | 0.5M NaOH | 3 |
| Storage | 0.1N NaOH | 3 |

### Ionic Exchange Chromatography, pH 5.0

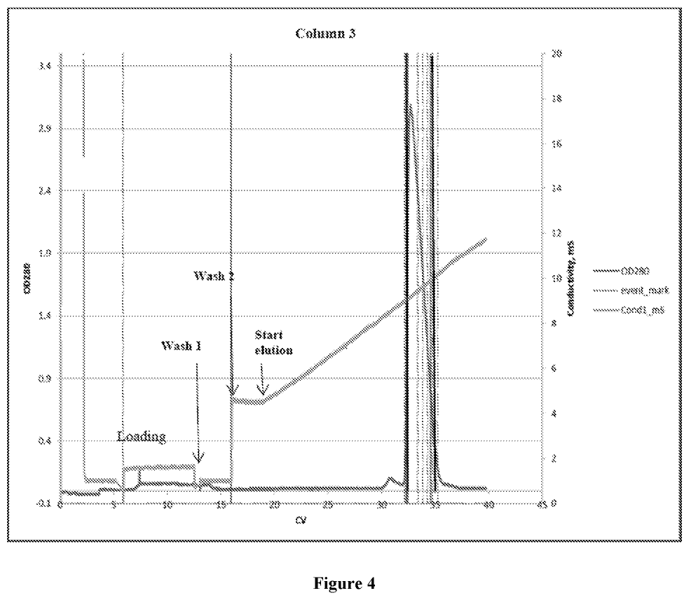

The eluate from the first CAPTO™ SP ImpRes ion exchange column was diluted with an equal volume of water for injection prior to loading onto a second CAPTO™ SP ImpRes ion exchange column equilibrated at a pH of about 5.0. The ranibizumab was bound to the second CAPTO™ SP ImpRes ion exchange column, and the column was developed using two isocratic washes. The product was then eluted using a gradient of increasing ionic strength. Table 3 provides a summary of the conditions for the ionic exchange chromatography step (at pH 5.0). FIG. 4 shows the real-time UV profile of the second ionic exchange column over a limited time window.

TABLE 3
CAPTO ™ SP ImpRes Chromatography Conditions, pH 5.0

| | | Step |
|---|---|---|
| | | Length |
| Process Step | Buffer | (CV) |
| Sanitization | 0.5M NaOH | 3 |
| Pre- | 200 mM NaCl, 20 mM acetate, 0.1 mM EDTA, | 3 |
| Equilibration | pH 5.0 | |
| Equilibration | 20 mM acetate, 0.1 mM EDTA, pH 5.0 | 3 |
| Load | Column 2 Capto SP ImpRes pool mixed with | NA |
| | equal volume water for injection | |
| Wash 1 | 20 mM acetate, 0.1 mM EDTA, pH 5.0 | 3 |
| Wash 2 | 80%, 20 mM acetate/0.1 mM EDTA/pH 5.0 | 5 |
| | 20%, 200 mM NaCl/20 mM acetate/0.1 mM | |
| | EDTA/pH 5.0 | |
| Elution | A buffer: 20 mM acetate, 0.1 mM EDTA, pH 5.0 | 33.3 |
| | B buffer: 200 mM NaCl, 20 mM acetate, 0.1 mM | |
| | EDTA, pH 5.0 | |
| | Linear gradient 20-70% B | |
| Strip | 200 mM NaCl, 20 mM acetate, 0.1 mM EDTA, | 3 |
| | pH 5.0 | |
| Sanitization | 0.5M NaOH | 3 |
| Storage | 0.1M NaOH | 3 |

### Phenyl Chromatography

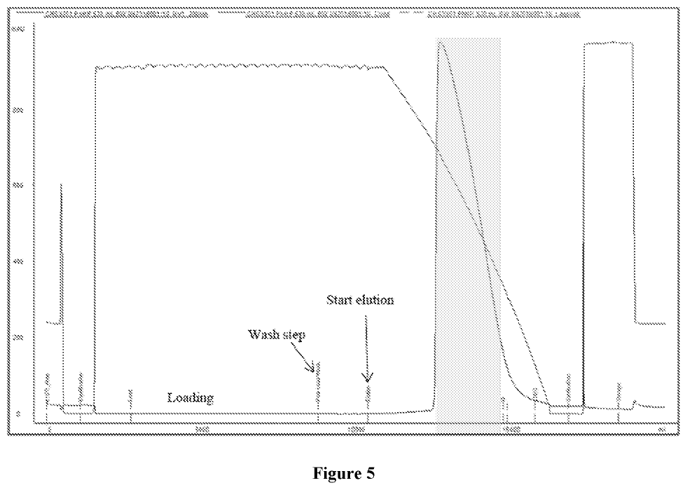

The primary function of the phenyl chromatography step was to remove product-related charged variants, host-cell impurities, and endotoxin. Prior to loading, the eluate from the pH 5.0 CAPTO™ SP ImpRes ion exchange column was conditioned to approximately 2.5 M NaCl. The product bound to the phenyl chromatography column and was eluted with a gradient of decreasing ionic strength. Table 4 provides a summary of the conditions for the phenyl chromatography. FIG. 5 shows the real-time UV profile of the phenyl column over a limited time window.

TABLE 4
Phenyl Chromatography Conditions

| | | Step |
|---|---|---|
| | | Length |
| Process Step | Buffer | (CV) |
| Sanitization | 0.5M NaOH | 3 |
| Equilibration | 50 mM Histidine, 2.5M NaCl, pH 5.5 | 3 |
| Load | Column 3 Capto SP ImpRes pool - conditioned | NA |
| Wash 1 | 50 mM Histidine, 2.5M NaCl, pH 5.5 | 6 |
| Elution | A buffer: 50 mM Histidine, 2.5M NaCl, pH 5.5 | 10 |
| | B buffer: 50 mM Histidine, pH 5.5 | |
| | Linear gradient 0-100% B | |
| Strip | water for injection | 3 |
| Sanitization | 0.5M NaOH | 3 |
| Storage | 0.1M NaOH | 3 |

### Ultrafiltration/Diafiltration

The purpose of this step was to concentrate the ranibizumab and to buffer exchange it into formulation buffer. The eluate from the phenyl column was conditioned by dilution with an equal volume of water for injection prior to ultrafiltration/diafiltration. The diluted eluate was then concentrated to approximately 12 mg/mL and diafiltered against formulation buffer lacking polysorbate. After diafiltration, the retentate was harvested from the tangential flow filtration (TFF) skid by filtration through a POSIDYNE® submicron filter.

### Example 2. Formulation of Ranibizumab

After removal from the TFF unit, the retentate was diluted to a first-dilution target protein concentration of 10.6 mg/mL using 10% (w/v) trehalose in 10 mM L-histidine, pH 5.5. The protein concentration was checked again and the retentate was further diluted to a target of 10.10 mg/mL using 10% (w/v) trehalose in 10 mM L-histidine, pH 5.5. The pool was conditioned to a level of 0.01% (v/v) polysorbate 20 by addition of 10 mL polysorbate spike solution/L retentate. The retentate was then asceptically filtered.

### Example 3. Yield and Purity of Ranibizumab

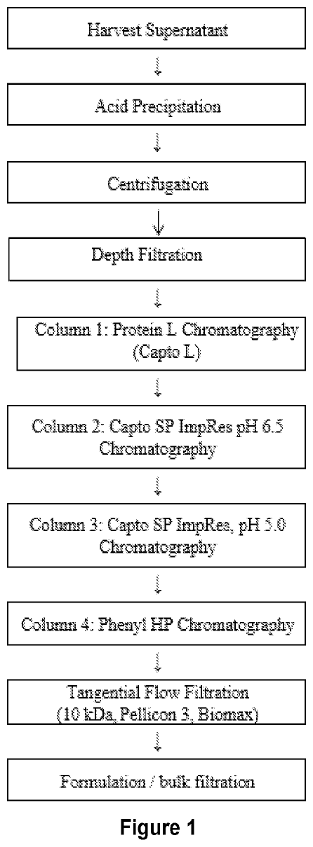

The purpose of this step was to determine the purity and yield of different lots of ranibizumab at different steps in the workflow described in FIG. 1. Table 5 shows a summary of the different methods used to assess purity and the results for each lot of ranibizumab.

TABLE 5
Purification Summary

| | | | | | nr-RP- | | |
|---|---|---|---|---|---|---|---|
| | | | SEC | CEX | HPLC | | |
| | Fermentation | | purity | Purity | purity | HCP | Endotoxin |
| Lot # | scale (L) | Stage | (%) | (%) | (%) | (ng/mg) | (EU/mg) |
| 1 | 500 | DS | 99.964 | 99.33 | 97.34 | <LOD | 1.7 |
| 2 | 500 | DS | 99.941 | 99.12 | 97.34 | 0.25 | 0.4 |
| | | | (99.9)** | (98.8)** | (96.9)** | | |
| 3 | 500 | DS | 99.749 | 99.094 | 97.489 | 0.37 | 2.0 |
| 4 | 20 | Column | 99.936 | 98.081 | >99.0 | <LOD | NT |
| | | 4 | | | | | |
| 5 | 20 | Column | 99.945 | 98.693 | 99.062 | <0.2 | NT |
| | | 4 | | | | | |
| 6 | 20 | Column | 99.872 | 98.413 | 99.079 | 0.178 | NT |
| | | 4 | | | | | |
| 7 | 20 | Column | 99.613 | 99.23 | NT | NT | NT |
| | | 3 | | | | | |
| 8 | 20 | Column | 99.41 | 94.046 | NT | NT | NT |
| | | 2 | | | | | |
| 9 | 20 | Column | 99.61 | 93.94 | NT | NT | NT |
| | | 2 | | | | | |
| 10 | 20 | Column | 99.21 | 95.974 | NT | NT | NT |
| | | 2 | | | | | |
| 11 | 20 | Column | 99.23 | 96.12 | NT | NT | NT |
| | | 2 | | | | | |
| Control #1 | | DP | 99.76 | 97.8 | 96.7 | NT | NT |
| Control #2 | | DP | 99.937 | 98.706 | >99.0* | NT | NT |

*No integratable peaks.
**Data in parentheses if a replicate of the assay.
DS: drug substance
DP: drug product
LOD: limit of detection
NT: not tested

In these experiments, Control #1 is Lucentis (an average of three different lots) and Control #2 is Lucentis.

In addition to measuring purity, purification yield was also measured at different steps during the process workflow described in FIG. 1. Table 6 shows purification yield summary for a subset of ranibizumab lots.

TABLE 6
Purification yield summary

| Sample | Lot # 4 | Lot # 5 | Lot # 6 | Lot # 7 | Lot # 3 | Lot # 2 | Lot # 1 |
|---|---|---|---|---|---|---|---|
| Broth AP- Titer-ProL | 0.353 | 0.298 | 0.365 | 0.312 | 0.271 | 0.22 | NT |
| (g/L) | | | | | | | |
| Column 1 - load (g/L) | 20.00 | 19.00 | 33.00 | 41.2 | — | — | — |
| Column 1 - load (g) | 0.457 | 0.431 | 0.749 | 0.936 | — | 59.4 | — |
| Column 1 - pool conc | 3.77 | 3.47 | 3.72 | 2.89 | — | 4.16 | — |
| (g/L) | | | | | | | |
| Column 1 - pool volume | 3.00 | 3.00 | 3.10 | 2.8 | — | — | — |
| (CV) | | | | | | | |
| Column 1 - protein (g) | 0.258 | 0.235 | 0.26 | 0.528 (2 | 9.160 | 31.45 | 52.398 |
| | | | | runs) | | | |
| Column 1 - Step yield | 56.45 | 54.5 | 34.80 | 54.8 | — | 52.95 | — |
| (%) | | | | | | | |
| Column 2 - load (g/L) | 10.00 | 15.00 | 15.00 | 15 | 8.29 | 8.19 | 13.0 |
| Column 2 - load (g) | 0.141 | 0.211 | 0.212 | 0.211 | 25.85 | 31.45 | 50.06 |
| Column 2 - pool conc | 0.95 | 1.60 | 1.57 | 1.3 | 0.62 | 0.677 | 0.84 |
| (g/L) | | | | | | | |
| Column 2 - pool volume | 2.50 | 2.97 | 3.00 | 2.48 | 3.05 | 2.35 | 3.58 |
| (CV) | | | | | | | |
| Column 2 - protein (g) | 0.033 | 0.067 | 0.077 | 0.0 46 | 5.89 | 6.11 | 11.55 |
| Column 2 - Step yield | 24.00 | 32.00 | 36.50 | 21.6 | 22.8 | 19.4 | 23.0 |
| (%) | | | | | | | |
| Column 3 - load (g/L) | 2.70 | 6.00 | 6.00 | 6.0 | 1.9 | 1.6 | 3.0 |
| Column 3 - load (g) | 0.015 | 0.034 | 0.034 | 0.034 | 5.89 | 6.11 | 11.55 |
| Column 3 - pool conc | 0.91 | 1.49 | 1.00 | 1.26 | 0.65 | 0.63 | 1.08 |
| (g/L) | | | | | | | |
| Column 3 - pool volume | 2.48 | 3.50 | 3.00 | 3.96 | 2.73 | 2.48 | 2.44 |
| (CV) | | | | | | | |
| Column 3 - protein (g) | 0.013 | 0.029 | 0.023 | 0.028 | 5.54 | 5.99 | 10.12 |
| Column 3 - Step yield | 85.00 | 86.00 | 68.00 | 83 | 93.0 | 98 | 87.6 |
| (%) | | | | | | | |
| Column 4 - load (g/L) | 6.60 | 7.80 | 5.84 | — | 9.0 | 11.1 | 6.1 |
| Column 4 - load (g) | 0.0213 | 0.025 | 0.019 | — | 3.53 | 5.42 | 9.8 (3 runs) |
| Column 4 - pool conc | 1.44 | 1.54 | 1.57 | — | 1.63 | 0.73 | 1.5 |
| (g/L) | | | | | | | |
| Column 4 - pool volume | 3.50 | 4.40 | 3.00 | — | 5.86 | 12.34 | 4.13 |
| (CV) | | | | | | | |
| Column 4 - protein (g) | 0.016 | 0.022 | 0.015 | — | 3.145 | 4.85 | 9.8 (3 runs) |
| Column 4 - Step yield | 75.10 | 87.50 | 78.90 | — | 89.2 | 89.5 | 100 |
| (%) | | | | | | | |

### Assessment of Purity and Yield at Different Stages of Purification

The purpose of this step was to assess quality of different lots of ranibizumab at different steps in the purification process. Table 7 shows a summary of the primary acidic variant, basic variant and main peak values of a CEX-HPLC assay for a subset of ranibizumab lots. Samples were taken after running ranibizumab through different chromatography columns in the process (as shown in FIG. 1).

TABLE 7
CEX-HPLC Assay

| | Acidic | Main | Basic |
|---|---|---|---|
| Sample | Peak % | Peak % | Peak % |
| Lot #1 (Eluate of Column 2) | 2.42 | 96.914 | 0.844 |
| Lot #2 (Eluate of Column 2) | 2.72 | 96.00 | 1.271 |
| Lot #3 (Eluate of Column 2) | 2.99 | 95.5 | 1.47 |
| Lot #1 (Eluate of Column 3) | 0.237 | 99.283 | 0.48 |
| Lot #2 (Eluate of Column 3) | 0.116 | 99.388 | 0.497 |
| Lot #3 (Eluate of Column 3) | 0.382 | 99.069 | 0.549 |
| Lot #1 (Eluate of Column 4) | 0.067 | 99.333 | 0.605 |
| Lot #2 (Eluate of Column 4) | 0.054 | 99.12 | 0.826 |
| Lot #3 (Eluate of Column 4) | 0.065 | 99.094 | 0.841 |
| Lucentis (Lot #S0082C) | 0.489 | 98.706 | 0.805 |

Table 8 shows a summary of the primary acidic variant, basic variant, and main peak values of a CEX-HPLC assay for a subset of ranibizumab lots after the second chromatography column (as shown in FIG. 1).

TABLE 8
CEX-HPLC Assay

| Sample | Acidic Peak % | Main Peak % | Basic Peak % |
|---|---|---|---|
| Lot #2 | 2.72 | 96.00 | 1.271 |
| Lot #3 | 2.99 | 95.5 | 1.47 |
| Lot #8 | 2.3 | 94.046 | 3.622 |
| Lot #9 | 2.44 | 93.94 | 3.617 |
| Lot #10 | 1.58 | 95.974 | 2.29 |
| Lot #11 | 1.578 | 96.12 | 2.256 |
| Lot #4 | 2.265 | 95.6 | 1.88 |
| Lot #5 | 1.724 | 97.116 | 1.14 |
| Lot #7 | 1.45 | 97.422 | 1.12 |
| Lot #12 | 2.786 | 94.566 | 2.648 |
| Lot #6 | 2.7 | 95.5 | 1.8 |

Table 9 shows the percentage of total eluted ranibizumab present in the high molecular weight peak (HMW), the main peak, and the low molecular weight peak (LMW) from the second, third, and fourth chromatography columns used in the process (as shown in FIG. 1) from experiments performed using three different lots of ranibizumab, and determined using the SEC-HPLC assay.

TABLE 9
SEC-HPLC Assay

| Sample | HMW % | Main Peak % | LMW % |
|---|---|---|---|
| Lot #1 (Eluate of Column 2) | 0.358 | 99.451 | 0.191 |
| Lot #2 (Eluate of Column 2) | 0.303 | 99.491 | 0.2 |
| Lot #3 (Eluate of Column 2) | 0.432 | 99.53 | 0.038 |
| Lot #1 (Eluate of Column 3) | * | 99.84 | 0.16 |
| Lot #2 (Eluate of Column 3) | 0.053 | 99.84 | 0.108 |
| Lot #3 (Eluate of Column 3) | 0.073 | 99.81 | 0.081 |
| Lot #1 (Eluate of Column 4) | 0.037 | 99.963 | 0.0 |
| Lot #2 (Eluate of Column 4) | 0.059 | 99.941 | 0.0 |
| Lot #3 (Eluate of Column 4) | 0.061 | 99.939 | 0.0 |

* Peak too small to integrate.

Table 10 shows the percentage of ranibizumab present in the main peak and the post main peak in an Nr RP-HPLC assay using the eluate of the fourth column in the purification process shown in FIG. 1 for three different ranibizumab lots.

TABLE 10
Nr RP-HPLC Assay

| Sample | Main Peak % | Post Main Peak % |
|---|---|---|
| Lot #1 | 97.340 | 2.66 |
| Lot #2 | 98.126 | 1.874 |
| Lot #3 | 97.489 | 1.523 |

Table 11 shows the level of HCP in ng/mL in the pooled eluate from each of the four chromatography columns used in the purification process (as shown in FIG. 1) for a subset of the ranibizumab lots. The limit of detection in the assay was 0.2 ng/mL and “NT” represents samples that were not tested. DS represents the final drug substance at the end of the purification process shown in FIG. 1. The limit of detection was 0.2 ng/mL and “NT” represents a sample that was not tested.

TABLE 11
HCP Assay

| | Fermentation | | Column 1 | Column 2 | Column 3 | Column 4 | |
|---|---|---|---|---|---|---|---|
| Lot | Scale | | Pool | Pool | Pool | Pool | DS |
| Lot #1 | 500 | L | 118.6 | 6.7 | <LOD | <LOD | <LOD |
| Lot #2 | 500 | L | 86.6 | 9.05 | <LOD | <LOD | 0.25 |
| Lot #3 | 500 | L | 56 | 16 | 8 | 1.5 | 0.37 |
| Lot #5 | 20 | L | 100 | 19 | 0.7 | <0.2 | |
| Lot #6 | 20 | L | NT | NT | <LOD | 0.178 | |
| Lot #7 | 20 | L | NT | NT | <LOD | <LOD | |
| Lot #4 | 20 | L | NT | NT | <LOD | <LOD | |

### Quality Assessment of Exemplary Ranibizumab Lot

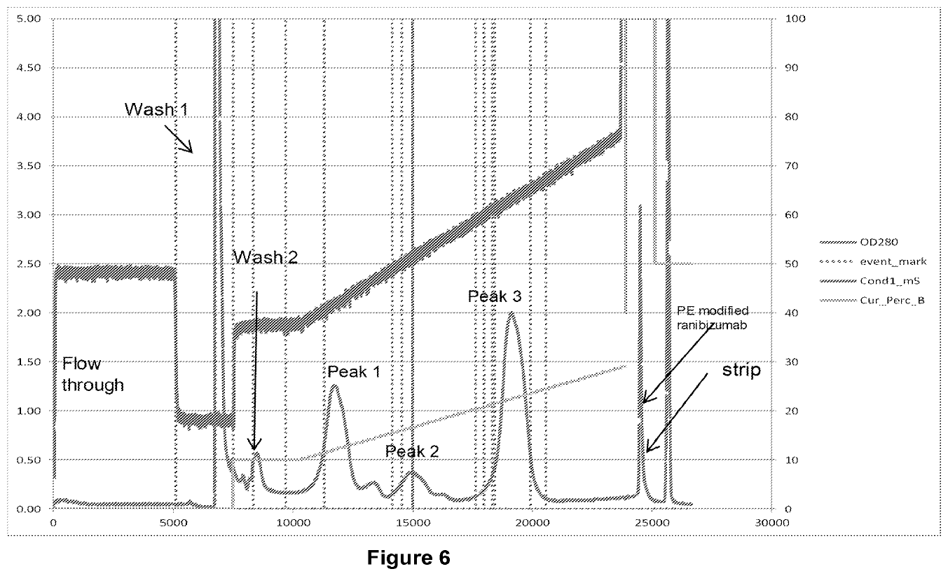

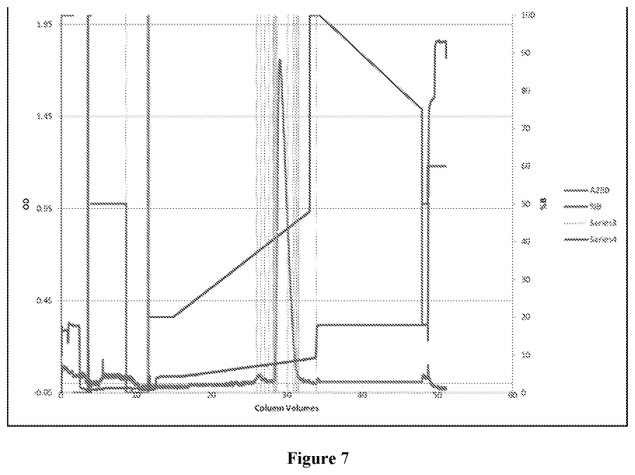

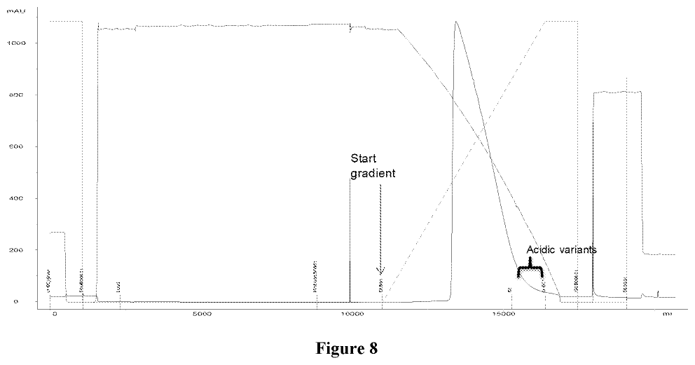

An exemplary ranibizumab lot (Lot #2) was assessed after being run through the second CAPTO™ SP ImpRes ion exchange column (pH 6.5) as described in the above Examples. The lot was processed on the column according to the methods described herein (e.g., same concentrations and volumes as described herein for the equilibration buffer, wash 1 buffer, wash 2 buffer, gradient buffer, and strip). FIG. 6 shows the UV profile and the resulting total protein harvested (6.11 g), which gave a 19.2% yield. The same lot (Lot #2) was assessed following purification through column 3 (Capto SP ImpRes, pH 5.0). The lot was processed on the column according to the methods described herein (e.g., same concentrations and volumes as described herein for the equilibration buffer, wash 1 buffer, wash 2 buffer, gradient buffer, and strip). FIG. 7 shows the UV profile following purification where 5.99 g was recovered, which gave a 98% yield. Next, the same lot (Lot #2) was assessed following purification through column 4 (Phenyl HP, pH 5.5). The lot was processed on the column according to the methods described herein (e.g., same concentrations and volumes as described herein for the equilibration buffer, wash 1 buffer, elution buffer, gradient buffer, and strip). FIG. 8 shows the phenyl chromatography step removed product-related charged variants, host-cell impurities, and endotoxins.

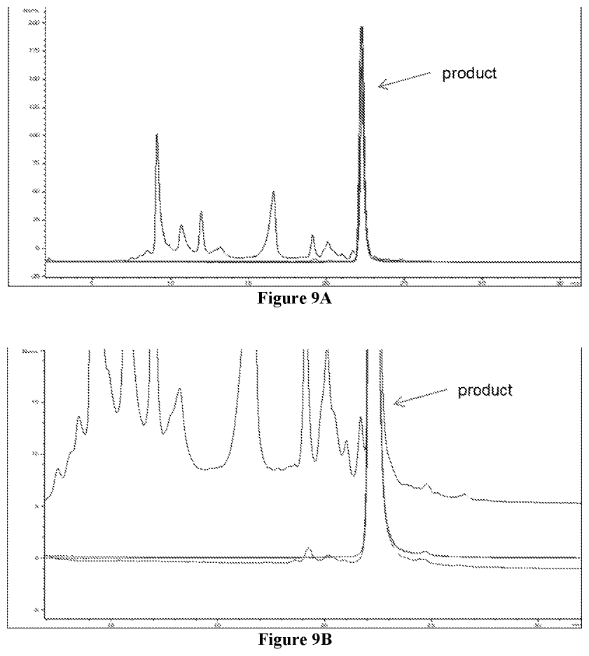

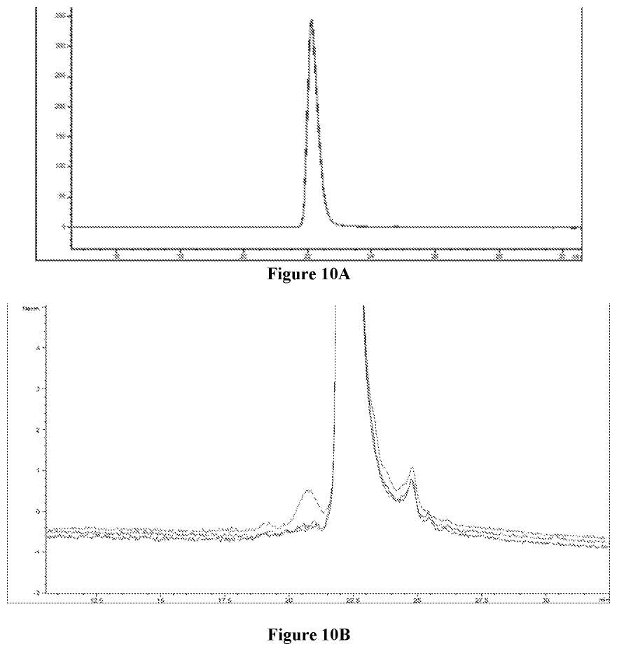

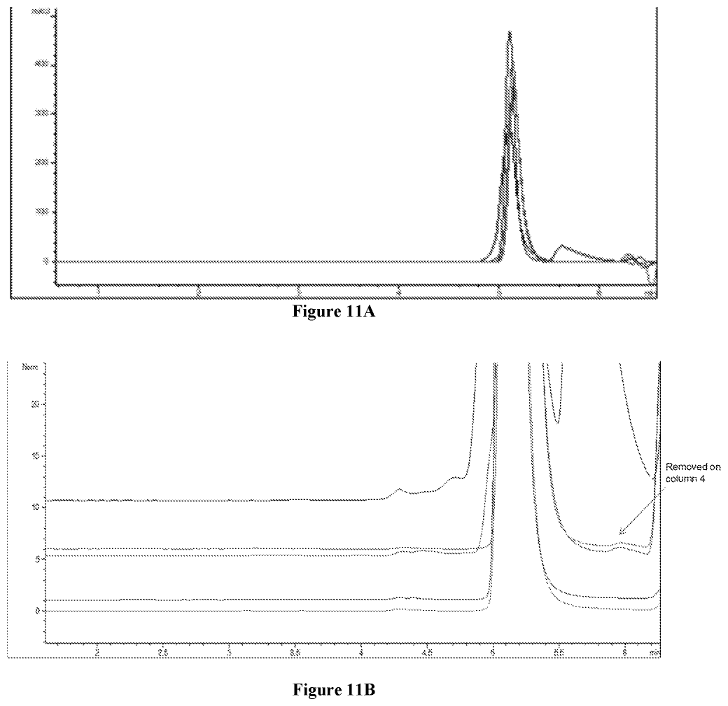

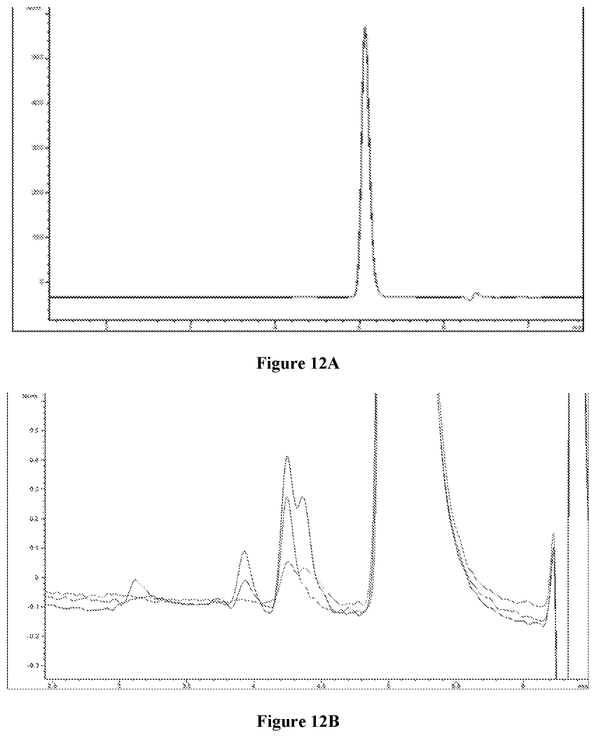

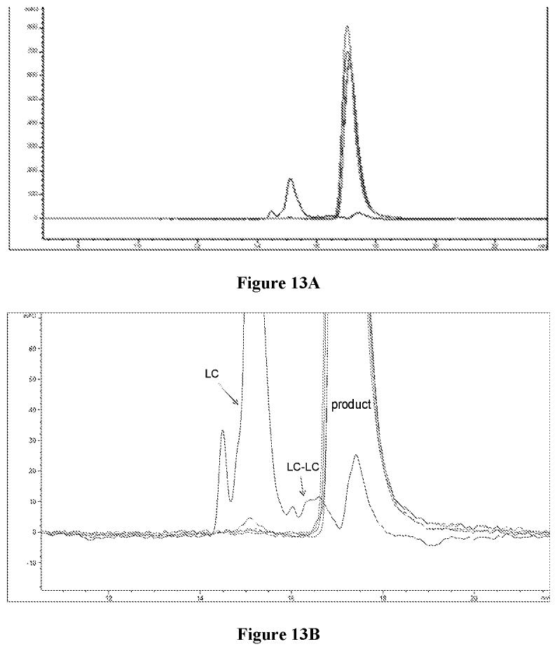

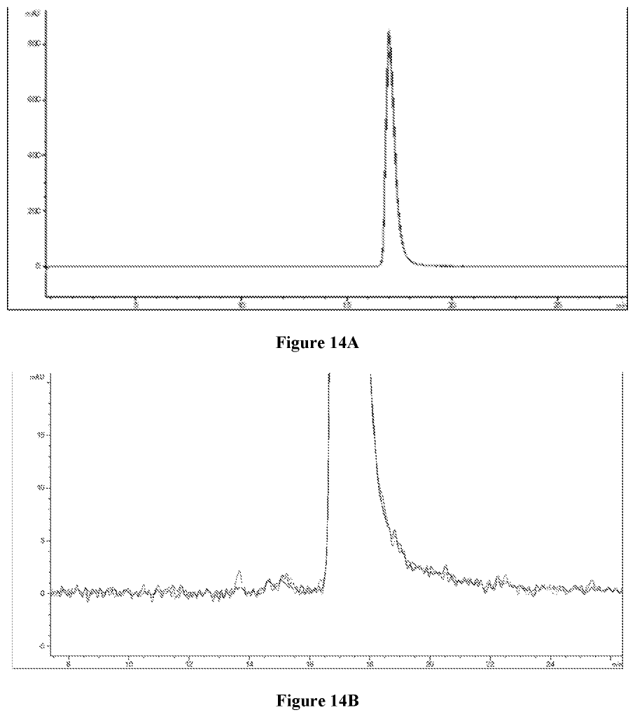

Lastly, CEX-HPLC (FIGS. 9A-9B and FIGS. 10A-10B), SEC-HPLC (FIGS. 11A-11B and FIGS. 12A-12B) and nrRP-HPLC (FIGS. 13A-13B and FIGS. 14A-14B) were used to assess purity.

Table 12 shows a summary of the purification for Lot #2 following each step in the workflow.

TABLE 12
Purification Summary for Exemplary Lot #2

| | Pooled fractions | Pool | Pool | Total | Step | |
|---|---|---|---|---|---|---|
| | Conc. A280 nm | volume | volume | Protein | Yield | Overall |
| Sample | (g/L) | (L) | (CV) | (g) | (%) | Yield (%) |
| AP Broth* | 0.22 titer | 270 | NA | 59.4 | ND | 100 |
| | assay) | | | | | |
| Column 1 | 4.16 | 7.623 | ND | 31.45 | 52.95 | 52.95 |
| (4 cycles- | | | | | | |
| 1 L column) | | | | | | |
| Column 2 | 0.677 | 9.025 | 2.35 | 6.11 | 19.4 | 10.2 |
| Column 3 | 0.63 | 9.514 | 2.48 | 5.99 | 98.0 | 10.1 |
| Column | 0.73 | 6.64 | 12.34 | 4.8472 | 89.5 | 8.14 |
| 4 ** | | | | | | |

The chromatogram of an SEC-HPLC assay performed on Lot #2 and Lot #3 shows that each successive step in the process eliminated molecules of incorrect size (e.g., aggregates) (FIGS. 11A and 11B). An SDS-PAGE gel of samples from Lot #2 was used to confirm the size of the purified samples and also confirm a reduction in molecules of incorrect size with each successive step in the process (data not shown). When compared to control ranibizumab, FIGS. 12A and 12B show similar SEP-HPLC profiles between the control and the samples (Lot #2 and Lot #3). Similar profiles between the control and samples (Lot #2 and #3) was also seen in nrRP-HPLC assays (FIGS. 14A and 14B).

### OTHER EMBODIMENTS

It is to be understood that while the disclosure has been described in conjunction with the detailed description thereof, the foregoing description is intended to illustrate and not limit the scope, which is defined by the appended claims. Other aspects, advantages, and modifications are within the scope of the following claims.

1. A process for manufacturing recombinant ranibizumab or a ranibizumab variant, the process comprising:
(a) providing a liquid comprising recombinant ranibizumab or a ranibizumab variant that is substantially free of cells;
(b) capturing the recombinant ranibizumab or ranibizumab variant in the liquid using an affinity chromatography column, wherein the eluate of the affinity chromatography column comprises the recombinant ranibizumab or the ranibizumab variant;
(c) purifying the recombinant ranibizumab or the ranibizumab variant in the eluate of step (b) using a first cation exchange chromatography column and buffers that have a pH of about pH 5.5 to about 7.5, wherein the eluate from the first cation exchange chromatography column comprises the recombinant ranibizumab or the ranibizumab variant; and
(d) purifying the recombinant ranibizumab or the ranibizumab variant in the eluate from the first cation exchange chromatography column using a second cation chromatography column and buffers that have a pH of about pH 4.0 to about pH 5.4, wherein the eluate from the second cation chromatography column comprises the recombinant ranibizumab or the ranibizumab variant.

2. The process of claim 1, wherein the liquid in (a) is a liquid culture medium that is substantially free of cells.

3. The process of claim 2, wherein the liquid culture medium is a liquid culture medium harvested from a culture of bacteria that secrete the recombinant ranibizumab or the ranibizumab variant.

4. The process of claim 3, wherein the culture of bacteria is a culture of E. coli that secrete the recombinant ranibizumab or the ranibizumab variant.

5. The process of claim 2, wherein the liquid culture medium has previously been subjected to acid precipitation.

6. The process of claim 2, wherein the method further comprises, prior to (a):
performing acid precipitation on the liquid culture medium to provide a precipitate comprising contaminants and the liquid comprising the recombinant ranibizumab or the ranibizumab variant of (a).

7. The process of claim 6, wherein the step of performing the acid precipitation on the liquid culture medium comprises adjusting the pH of the liquid culture medium to about 3.5 to about 4.5 or to about 3.8 to about 4.2.

8. (canceled)

9. The process of claim 6, wherein the step of performing the acid precipitation on the liquid culture medium comprises the use of centrifugation.

10. The process of claim 1, wherein the method further comprises between (a) and (b):
filtering the liquid comprising the recombinant ranibizumab or the ranibizumab variant.

11. The process of claim 1, wherein the capturing is performed using an antibody- or antibody fragment-binding capture mechanism.

12. The process of claim 11, wherein the antibody- or antibody-fragment binding capture mechanism is a kappa light chain-binding capture mechanism.

13. The process of claim 12, wherein the affinity chromatography column comprises an immobilized Protein L chromatography resin.

14. The process of claim 1, wherein the step of purifying the recombinant ranibizumab or the ranibizumab variant in the eluate of step (b) using the first cation exchange chromatography column is performed using buffered solutions having a pH of about 6.2 to about 6.8 or a pH of about 6.5.

15. (canceled)

16. The process of claim 1, wherein the first cation exchange chromatography column comprises a S or SP chromatography resin.

17. The process of claim 1, wherein the method further comprises between (c) and (d):
adjusting ionic concentration of the eluate of the first cation exchange chromatography column.

18. The process of claim 1, wherein the method further comprises between (c) and (d):
diluting the eluate of the first cation exchange chromatography column.

19. The process of claim 1, wherein the step of purifying the recombinant ranibizumab or the ranibizumab variant in the eluate from the first cation exchange chromatography column using the second cation exchange chromatography column is performed using buffered solutions having a pH of about 4.5 to about 5.4, a pH of about 4.8 to about 5.2, or a pH of about 5.0.

20-21. (canceled)

22. The process of claim 1, wherein the second cation exchange chromatography column comprises a S or SP chromatography resin.

23. The process of claim 1, further comprising after (d):
filtering the eluate from the second cation exchange chromatography column.

24. The process of claim 1, further comprising after (d):
adjusting ionic concentration of the eluate of the second cation exchange chromatography column.

25. The process of claim 1, further comprising after (d):
diluting the eluate of the second cation exchange chromatography column.

26. The process of claim 1, further comprising after step (d):
purifying and polishing the recombinant ranibizumab or the ranibizumab variant in the eluate from the second cation exchange chromatography column using a hydrophobic interaction chromatography column, wherein the eluate of the hydrophobic interaction chromatography column comprises the recombinant ranibizumab or the ranibizumab variant.

27. The process of claim 26, wherein the hydrophobic interaction chromatography column comprises a phenyl sepharose chromatography resin.

28. The process of claim 27, further comprising filtering the eluate from the hydrophobic interaction chromatography column.

29. The process of claim 1, further comprising formulating the ranibizumab or the ranibizumab variant into a pharmaceutical composition.

30. A pharmaceutical composition produced by the method of claim 29.

31. A kit comprising the pharmaceutical composition of claim 30.

32. A method of treating a subject in need thereof that comprises administering to the subject a therapeutically effective amount of the pharmaceutical composition of claim 30.


SEQUENCE LISTING (20 sequences)

SEQ ID NO: 1 - protein, 110 aa, Artificial
  Ranibizumab Light Chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 2 - protein, 158 aa, Artificial
  Ranibizumab Heavy Chain
SGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 3 - protein, 214 aa, Artificial
  Ranibizumab Light Chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 235 aa, Artificial
  Ranibizumab Heavy Chain
EVQLVESGGGLVQPGGSLRLSCAASGYDFTHYGMETNWVRQAPGKGLEWVGWINTYTGEPTYAADFKRRFTFSLDTSKSTAYLQMETNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 22 aa, Artificial
  Signal Sequence
MKYLLPTAAAGLLLLAAQPAMA

SEQ ID NO: 6 - protein, 21 aa, Artificial
  Signal Sequence
MKKTAIAIAVALAGFATVAQA

SEQ ID NO: 7 - protein, 23 aa, Artificial
  Signal Sequence
MKKNIAFLLASMFVFSIATNAYA

SEQ ID NO: 8 - protein, 28 aa, Artificial
  Signal Sequence
MFKFKKKFLVGLTAAFMSISMFSATASA

SEQ ID NO: 9 - protein, 21 aa, Artificial
  Signal Sequence
MKQSTAILALLPLLFTPVTKA

SEQ ID NO: 10 - protein, 22 aa, Artificial
  Signal Sequence
MMKRNILAVIVPALLVAGTANA

SEQ ID NO: 11 - protein, 21 aa, Artificial
  Signal Sequence
MKKSTLALVVMGIVASASVQA

SEQ ID NO: 12 - protein, 26 aa, Artificial
  Signal Sequence
MKIKTGARILALSALTTMMFSASALA

SEQ ID NO: 13 - protein, 21 aa, Artificial
  Signal Sequence
MKVKVLSLLVPALLVAGAANA

SEQ ID NO: 14 - protein, 20 aa, Artificial
  Signal Sequence
MKATKLVLGAVILGSTLLAG

SEQ ID NO: 15 - protein, 25 aa, Artificial
  Signal Sequence
MMITLRKLPLAVAVAAGVMSAQAMA

SEQ ID NO: 16 - protein, 20 aa, Artificial
  Signal Sequence
MRAKLLGIVLTTPIAISSFA

SEQ ID NO: 17 - protein, 23 aa, Artificial
  Signal Sequence
MSIQHFRVALIPFFAAFCLPVFA

SEQ ID NO: 18 - protein, 21 aa, Artificial
  Signal Sequence
MKQSTIALALLPLLFTPVTKA

SEQ ID NO: 19 - protein, 25 aa, Artificial
  Signal Sequence
MKVMRTTVATVVAATLSMSAFSVFA

SEQ ID NO: 20 - protein, 23 aa, Artificial
  Signal Sequence
MKKSLVLKASVAVATLVPMLSFA